(19) United States
(12) Patent Application Publication
(10) Pub. No.: US 2013/0058918 A1
(43) Pub. Date: Mar. 7, 2013

(54) ISOLATION AND USE OF A NEW TYPE OF GLIAL CELL WITH NEUROTOXIC POTENTIAL

(71) Applicants: Luis Barbeito, Montevideo (UY); Pablo Diaz-Amarilla, Montevideo (UY); Silvia Olivera, Montevideo (UY); Javier Ganz, Montevideo (UY)

(72) Inventors: Luis Barbeito, Montevideo (UY); Pablo Diaz-Amarilla, Montevideo (UY); Silvia Olivera, Montevideo (UY); Javier Ganz, Montevideo (UY)

(73) Assignee: Cedars-Sinai Medical Center, Los Angeles, CA (US)

(21) Appl. No.: 13/696,971

(22) PCT Filed: May 11, 2011
(86) PCT No.: PCT/IB11/00997

§ 371 Date: Nov. 8, 2012

Related U.S. Application Data
(60) Provisional application No. 61/334,103, filed on May 12, 2010.
(60) Provisional application No. 61/416,706, filed on Nov. 23, 2010.

(51) Int. Cl. C12N 5/10; G01N 21/64; A61P 25/28; G01N 27/62; A61K 39/395; G01N 21/76; G01N 33/566

(52) U.S. Cl. 424/130.1; 435/353; 435/7.9; 435/7.1; 435/29

(57) ABSTRACT

The present invention provides for an isolated population of aberrant astrocytes, methods of isolating the aberrant astrocytes, methods of diagnosing neurodegenerative diseases, methods of drug screening using the aberrant astrocytes, and treatment methods targeting function, activity, and signaling associated with aberrant astrocytes.

### FIELD OF THE INVENTION

This invention relates to diagnostics, therapeutics and drug screening methods for neurodegenerative diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The causes of progressive loss of upper and lower motor neurons in Amyotrophic Lateral Sclerosis (ALS) remain largely unknown. Until recently, research on ALS pathogenesis has mainly focused on motor neuron degeneration; the concomitant pathological changes in other cell types including glial cells have been mostly neglected. Neurodegeneration in ALS is associated with activation and proliferation of glial cells surrounding damaged motor neurons from the motor cortex, brain stem and the spinal cord. In the animal models of ALS expressing SOD1 mutations, there is increased proliferation of astrocytes, microglia as well as glial progenitor cells, generating a variety of glial phenotypes. Exaggerated inflammatory response of glial cells occurring after disease onset is thought to influence the progression of motor neuron degeneration. Genetic excision of mutant SOD1 in astrocytes or microglia extend disease duration in ALS mice, with no effect on disease onset. (Yamanaka K, et al. (2008) Mutant SOD1 in cell types other than motor neurons and oligodendrocytes accelerates onset of disease in ALS mice. Proc Natl Acad Sci 105: 7594-7599; Boillee S, et al. (2006) ALS: a disease of motor neurons and their normeuronal neighbors. Neuron 52: 39-59.) Taken together, these studies suggest a scenario of motor neurons being the main target of SOD1 mutations and glial cells modulating the rate of degeneration.

Cultured astrocytes bearing the SOD1^{G93A} mutation can induce apoptosis of nontransgenic embryonic motor neurons cultured on the top of the glia. (Vargas M R, et al. (2006) Increased glutathione biosynthesis by Nrf2 activation in astrocytes prevents p75NTR-dependent motor neuron apoptosis. J Neurochem 97: 687-696.) These observations were confirmed and further characterized by other independent studies. In another study, motor neurons obtained by differentiation of mouse embryonic stem cells and survival of these cells decreased when maintained in coculture with glial cells expressing mutant SOD1. (Di Giorgio FP, et al. (2007) Non-cell autonomous effect of glia on motor neurons in an embryonic stem cell-based ALS model. Nat Neurosci 10: 608-614.) Others have reported that mutant astrocytes and their conditioned media induced apoptosis of spinal primary and embryonic mouse stem cell-derived motor neurons, indicating toxicity is mediated by soluble factors. (Nagai M, et al. (2007) Astrocytes expressing ALS-linked mutated SOD1 release factors selectively toxic to motor neurons. Nat Neurosci 10: 615-622.) More recent studies have shown the toxicity of mutant astrocytes could also affect motor neurons derived from human embryonic stem cells. (Di Giorgio FP, et al. (2008) Human embryonic stem cell-derived motor neurons are sensitive to the toxic effect of glial cells carrying an ALS-causing mutation. Cell Stem Cell 3: 637-648; Marchetto M C, et al. (2008) Noncell-autonomous effect of human SOD1 G37R astrocytes on motor neurons derived from human embryonic stem cells. Cell Stem Cell 3: 649-657.)

Since there are no available treatments to cure these diseases and current diagnosis is mainly performed after the onset of clinical symptoms, at time where most motor neurons are already lost, there exists a need in the art for diagnostic approaches that permit early diagnosis or prognosis, as well as new therapeutic strategies to stop or delay the disease progression.

### SUMMARY OF THE INVENTION

The following embodiments and aspects thereof are described and illustrated in conjunction with compositions and methods which are meant to be exemplary and illustrative, not limiting in scope. The present invention provides an isolated cell having the ability to induce neuron death, wherein the isolated cell can express at least one of the markers selected from the group consisting of GFAP, nestin, vimentin, S100β, CX43, GLAST, GLT1, glutamine synthase, Ng2, Olig2, A2B5, ED1, and combinations thereof. In one embodiment, the isolated cell can express at least one of the markers selected from the group consisting of GFAP, S100β, CX43, and combinations thereof. In another embodiment, the isolated cell can produce one or more secretory factors to induce the neuron death. In another embodiment, the isolated cell can secrete the one or more secretory factors into media to condition the media. In another embodiment, the conditioned media can exhibit at least 100 times more toxic activity to induce neuron death when compared to conditioned media from normal astrocytes. In another embodiment, the isolated cell can expand in culture for at least 1 passage. In another embodiment, the isolated cell can expand in culture for at least 20 passages. In another embodiment, the isolated cell can proliferate at a rate about two times that of normal astrocytes. In another embodiment, the isolated cell can be isolated from a mammal. In another embodiment, the mammal can have a SOD1G93 mutation. In another embodiment, the SOD1G93 mutation can be a SOD mutation. In a particular embodiment, the mammal can be a transgenic rat having a SOD1G93A mutation. The present invention also provides a culture comprising an isolated cell having the ability to induce neuron death, wherein the isolated cell can express at least one of the markers selected from the group consisting of GFAP, nestin, vimentin, S100β, CX43, GLAST, GLT1, glutamine synthase, Ng2, Olig2, A2B5, ED1, and combinations thereof.

Another embodiment of the present invention provides a method of isolating cells having the ability to induce neuron death, comprising obtaining a tissue sample from a mammal, disaggregating the tissue sample to extract cells, plating the extracted cells on a culture surface and culturing the cells on the culture surface in a culture media, wherein the cells having the ability to induce neuron death can be isolated and can express at least one of the markers selected from the group consisting of GFAP, nestin, vimentin, S100β, CX43, GLAST, GLT1, glutamine synthase, Ng2, Olig2, A2B5, ED1, and combinations thereof. In another embodiment, the isolated cells can express at least one of the markers selected from the group consisting of GFAP, S100β, CX43, and combinations thereof. In another embodiment, the isolated cells can produce one or more secretory factors to induce the neuron death. In another embodiment, the isolated cells can expand in culture for at least 1 passage. In another embodiment, the isolated cells can expand in culture for at least 20 passages. In another embodiment, the isolated cell can proliferate at a rate about two times that of normal astrocytes. In another embodiment, a solid substrate can be placed in contact with the isolated cells. In another embodiment, the solid substrate can be agarose. In another embodiment, the tissue sample can comprise motor cortex, brain stem, spinal cord, or skeletal muscle tissue from a mammal. In another embodiment, the mammal can have a SOD1G93 mutation. In another embodiment, the SOD1G93 mutation can be a SOD1G93 mutation. In another embodiment, the mammal can be a transgenic rat having a SOD1G93A mutation.

Another embodiment of the present invention provides a method of treating a neurodegenerative disease comprising selecting a patient in need of treatment for a neurodegenerative disease and administering a compound capable of modulating the activity of an aberrant astrocyte to treat the subject for the neurodegenerative disease.

Another embodiment of the present invention provides a method of detecting a neurodegenerative disease comprising obtaining a sample from a subject suspected of having a neurodegenerative disease, measuring the level of a protein produced by an aberant astrocyte and comparing the level of the protein from the subject suspected of having a neurodegenerative disease to the level of the protein in a subject without the neurodegenerative disease, wherein a difference in the level of the protein can detect the neurodegenerative disease. In another embodiment, the difference in the level of the protein can be a statistically significant difference.

Another embodiment of the present invention provides a method of identifying a compound capable of modulating aberrant astrocyte activity, comprising providing a population of aberrant astrocytes, contacting the population of aberrant astrocytes with a test compound, measuring a parameter of the population of aberrant astrocytes, wherein the parameter is selected from the group consisting of proliferation, cytotoxicity, alterations in a protein function, or alterations in a protein expression, and combinations thereof, and comparing the measured parameter from the population of aberrant astrocytes contacted with the test compound to a measured parameter from a population of aberrant astrocytes not contacted with the test compound, wherein a difference in the measured parameter can identify the compound capable of modulating aberrant astrocyte activity. In another embodiment, the difference in the measured parameter can be a statistically significant difference.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts microphotographs showing the morphology of cultured cells in accordance with various embodiments of the present invention. Note the increase in cell number in the culture of the symptomatic rat spinal cord. SOD1^{G93A} rats (Tg) were sacrificed when showing signs of paralysis (symptomatic stage, 180 days old), and were compared to non-transgenic litter mates of the same age (No-Tg). The spinal cord was dissected, cut in pieces and trypsinized. The cells were then plated in a culture flask and observed at 1, 5 and 16 DIV (days in vitro), under a phase contrast microscope. From 5-16 DIV an agarose layer was polymerized on the top of the cells.

FIG. 2 depicts the establishment of AbA cells cultures in accordance with various embodiments of the present invention. Primary cultures of the spinal cord were prepared from symptomatic SOD1^{G93A} rats or non transgenic littermates. A: The cells were plated in a 25 cm² culture flask and observed at 2DIV (days in vitro), 7DIV and 16 DIV under a phase contrast microscope. Note the increase in cell number in the culture of the symptomatic rat spinal cord as compared to age-matched non-transgenic litter mates (insert in upper panels). Arrows indicate microglial cells. Asterisks and Starts indicate polygonal and proliferative elongated astrocytes, respectively. Scale bar=50 μm. B: Identification of cell types in 16 DIV cultures of SOD1^{G93A} rats by immunocytochemistry to GFAP (red), S100β (green) and ED1. Scale bar=μm. C: Representative morphology of a confluent AbA cell culture at passage 12 in phase contrast microscope. Scale bar=50 μm.

FIG. 3 depicts expression of astrocyte markers and proliferation in AbA cells in accordance with various embodiments of the present invention. Cultures of AbA cells (passage 12) were compared to non-transgenic or SOD1^{G93A} transgenic primary neonatal spinal cord astrocytes. A: Confocal imaging of cells immunostained to determine expression of GFAP, S100β, vimentin, glutamine synthase (GS) and Cx43. Note pretreatment with 10 uM forskolin (FKL) for 6 h dramatically increased GFAP staining and process growth in both AbA and primary astrocytes. Scale bar=50 μm: Western blot analysis of astrocytic markers in AbA cells as compared to primary astrocytes (GFAP=glial acidic fibrillary protein, Cx43=connexin 43, GLT-1=glutamate transporter, GS=glutamine synthase, β-Actin) C: Western blot analysis of intracellular and secreted SOD1^{G93A} levels in AbA and primary astrocytes. D: Growth of AbA cells or primary astrocytes assessed for 9 consecutive days. At each time point, cells were lysed and intact cell nuclei were counted with a hemacytometer. E: Fluorescent microscopy of cells counted in sub-FIG. 4D, demonstrating high proliferation of AbA cells in comparison to transgenic (Tg) and non-transgenic (No-Tg) cells.

FIG. 4 depicts phenotypic characterization of AbA cells isolated from the spinal cord of symptomatic SOD1^{G93A} rats in accordance with various embodiments of the present invention. Compared to primary cultures of neonatal astrocytes from non-transgenic and transgenic rats, A: AbA cells form a monolayer of cells mainly expressing S100 beta (green) with reduced amounts of GFAP (red) in comparison to non-transgenic (non-Tg) and transgenic (SOD1G93A) cells. B: In addition, AbA cells express high levels of Connexin 43, presenting a patchy distribution in the surface of the cells. C: In western blot analysis, AbA cells express higher Cx43 but low levels of GFAP and blunted levels of GLT1.

FIG. 5 depicts AbA cells displaying astrocyte-like GFAP processes when treated with Forskolin during 3 h in accordance with various embodiments of the present invention. The effect of Forskolin (Fk) is similar than that observed in non-transgenic and transgenic primary astrocyte cultures.

FIG. 6 depicts identification of AbA-like cells in the degenerating spinal cord in accordance with various embodiments of the present invention. A: GFAP (red) and S100β (green) immunostaining of lumbar spinal cord sections from non-transgenic (non-Tg), asymptomatic (Tg-Asymp) and symptomatic SOD1^{G93A} rats (Tgsymp). Dotted lines in upper panels indicate the limit between grey and white matter. The surface of large motoneurons has been designed in lower panels. Note S100β was upregulated in the spinal cord of symptomatic rats and particularly expressed in a population of hypertrophic cells with astrocyte morphology. Most of these cells displayed co-localization (yellow) of S100β and GFAP. Cx43 immunoreactivity was also dramatically increased in Tg-symp spinal cords, being colocalized with S100b in most perineuronal hypertrophic AbA-like cells (yellow in lower panel). Scale bar=50 μm, except Cx43 Scale bar=20 μm. B: BrdU immunostaining of spinal cord sections from symptomatic SOD1^{G93A} rats. Note the labeling of nuclei in apparent glial cells expressing S100b or Cx43 that surround damaged motoneurons. Scale bar=50 μm. C: Double staining for proliferation marker Ki67 and S100b demonstrates active proliferation near degenerating motor neurons.

FIG. 7 depicts immunohistochemistry of GFAP and S100 beta in the ventral cord of rat lumbar cord in accordance with an embodiment of the present invention. Note the increased expression of S100 beta in a subpopulation of cells also expressing GFAP in symptomatic SOD1^{G93A} rats.

FIG. 8 depicts AbA cells mediating motor neuron death by secreting soluble factors in accordance with various embodiments of the present invention. A: Embryonic rat motor neurons were seeded on the top of primary astrocytes cultures from non-transgenic (no-TG), transgenic rats (TG) or AbA cell monolayer. Neuronal survival was assessed 72 h later. Note that survival of motor neurons was below to 10% as compared with No-TH (100%). *p<0.05 with respect to ACM from non-transgenic astrocytes. B: increasing dilutions of conditioned media prepared from No-TG, TG or AbA cells was used to culture motor neurons seeded on laminin/polyomithine substrate. Dilution series are labeled with E1 denoting 1/10 dilution, E2 denoting 1/100 dilution, E3 denoting 1/1000 dilution, etc. Note the conditioned media from AbA cells exerted significant neuronal loss up to dilutions of 1:10,000 dilution, suggesting toxicity was mediated by a diffusible factor.

FIG. 9 shows that AbA cells mediate motor neuron death by secreting soluble factors in accordance with various embodiments of the present invention. A: Embryonic rat motor neurons were seeded on the top of confluent feeder layers of primary astrocytes cultures from non-transgenic (no-TG), transgenic rats (TG) or AbA cells. Neuronal survival was assessed 72 h later. Note that survival of motor neurons was <10% as compared with non-transgenic (100%, dotted line) and transgenic astrocytes. *p<0.05 with respect to non-transgenic astrocytes. B: Astrocyte conditioned media (ACM) prepared from No-TG, TG or AbA cells were added to pure motor neurons seeded on laminin/polyomithine substrate. The final fold dilution is indicated in each condition. Note, conditioned media from AbA cells exerted significant neuronal loss up to 1:10000 dilution. *p<0.05 with respect to ACM from non-transgenic astrocytes. C: The neurotoxic activity of increasing dilutions of ACM was assessed in primary cultures of hippocampal neurons.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Isolated” as used herein with reference to a population of cells refers to cells that are isolated or purified relative to other cell types in the source material. For example, “isolated population of aberrant astrocytes” refers to a population of aberrant astrocytes that is isolated relative to other types of cells, such as normal astrocytes. In various embodiments, an isolated population of cells (e.g., aberrant astrocytes) will contain at least 60%, 65%, 70%, 75%, 80%, 85%, 90%, 95%, 96%, 97%, 98%, or 99% of the cells of interest (e.g., aberrant astrocytes) relative to other types of cells.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Differentiation” as used in the present invention means the change of cells capable of forming multiple cell types into cells capable of forming fewer cell types, committed to a specific lineage and/or cells having characteristic functions, such as microglia, astrocytes, oligodendrocytes, ependymal cells, radial glia, Schwann cells, satellite cells, and enteric glial cells.

“Treatment” or “treating” refers to therapy, prevention or prophylaxis and particularly refers to the administration of medicine or the performance of medical procedures with respect to a subject. Treatment may be for prophylactic purposes to reduce the extent or likelihood of occurrence of a disease state, disorder or condition. Treatment may also be for the purpose of reducing or eliminating symptoms of an existing disease state, disorder, condition, or undesirable appearance. Treatment may directly eliminate infectious agents or other noxious elements causing a disease state, disorder or a condition. Treatment may alternatively occur through enhancement and stimulation of an organism's natural immune system, such as promoting or facilitating repair and regeneration of damaged or disease cells and/or tissue. Treatment may also occur by supplementing or enhancing the body's normal function.

“Subject” or “patient” refers to a mammal, including a human, in need of treatment for a condition, disorder or disease.

“Neurodegenerative disease” refers to a disease or condition associated with diminished structure or function of the central, peripheral, and enteric nervous systems. Examples include: Alzheimer's disease, Frontotemporal dementia, Prion disorders, Parkinson's disease, Dementia with Lewy bodies, Corticobasal degeneration, Progressive supranuclear palsy, Huntington's disease, Multiple system atrophy, Amyotrophic lateral sclerosis, Spinal muscular atrophy, Hereditary spastic paraparesis, Spinocerebellar atrophies, Friedreich's ataxia, Amyloidoses, Multiple Sclerosis, Charcot Marie Tooth, among others.

Amyotrophic lateral sclerosis (abbreviated ALS, also referred to as Lou Gehrig's disease) is a motor neuron disease caused by the degeneration of neurons located in the ventral horn of the spinal cord and the cortical neurons that provide their afferent input. The disorder, which manifests itself in sporadic and familial forms, is characterized by rapidly progressive weakness, muscle atrophy and fasciculations, spasticity, dysarthria, dysphagia, and respiratory compromise. Although a leading view hypothesizes that pathogenesis of ALS is driven by motor neuron damage as a consequence of direct oxidative stress, there is now substantial evidence that non-neuronal cells also play a key role in the disease. Familial studies and animal models have contributed to the growing body of evidence suggesting that events occurring outside of motor neurons are critical steps in progression of the disease. (Carter B J et al. (2009) Redox modifier genes and pathways in amyotrophic lateral sclerosis. Antioxidants & Redox Signaling 11:1569-1586.)

The role of oxidation in ALS disease progression has been advanced by studies of familial forms of ALS (fALS) revealing an important role for superoxide dismutase 1 (SOD1) in pathogenesis of the disease. To date, over 150 mutations in the coding sequence of SOD1 have been identified in ALS patients. This enzyme catalyzes the detoxification of superoxide anion (O₂⁻) to molecular oxygen and hydrogen peroxide (2O₂⁻+2H⁺→O₂+H₂O₂). ALS patients are known to suffer from unusually high levels of oxidative protein damage, as demonstrated by protein carbonyl and nitrotyrosine modifications in diseased spinal cord measurements. However, it is not clear if redox-stress is a primary defect or a secondary consequence of the disease in sporadic ALS. Further aiding our understanding of redox-stress in ALS is the development of animal models replicating features of fALS. In particular, a common form of SOD1 mutation arises from substitutions occurring at the G93 position of the in the short loop V of the protein which connects anti-parallel β-strands that forms part of the homodimeric SOD1 protein and is near residues 37-42 of loop III. Six pathogenic substitutions are known to exist at the G93 location (G93A, G93C, G93D, G93R, G93S, and G93V). It is believed that these substitutions destabilize the β-barrel by interfering with packing of residues that form the “apolar plug” found at one end of the β-barrel. (Galaleldeen A et al. (2009) Structural and biophysical properties of metal-free pathogenic SOD1 mutants A4V and G93A. Arch Biochem Biophys 492: 40-47.) This change in protein structure reduces or eliminates copper and zinc ions from binding to the protein and interferes with the vital role of SOD1 in detoxification of superoxide, which is present in large amounts in highly respiring tissues such as brain and spinal cord.

Transgenic animals, such as mice and rats, harboring the G93A mutation in SOD1 have provided a valuable model for evaluating the specific role of this molecule in ALS pathogenesis. Further, a variety of studies evaluating features of these transgenic animals has focused on the role of glial cells in furthering ALS-like disease progression. In normal adult brain, glial cells such as microglia and astrocytes play an important role in development, blood flow regulation, homeostasis, synaptic function, among many others. (Papadimitriou D et al. (2010) Inflammation in ALS and SMA: sorting out good from the evil. Neurobiol Dis 37:493-502.) However, in a variety of neurodegenerative diseases and conditions, neuroinflammation is present in the form of “gliosis” (sometimes called “reactive astrocytosis”), wherein locations of neuroinflammation is marked by the presence of activated astrocytes and microglia. (Wang DD and Bordey A. (2008) The astrocyte odyssey. Prog Neurobiol 86:342-367.) Characterization of these activated astrocytes and microglia is crude and imprecise, given that the chief detection method relies on increased immunoreactivity for the intermediate filament, glial fibrillary acid protein (GFAP). (Sofroniew M V and Vinter H V. (2010) Astrocyte: biology and pathology. Acta neuropathol 119:7-35.) The presence of increasing reactive GFAP may be unrelated to the underlying causes of ALS disease progression itself, but instead may simply demonstrate increased stainability of tissue, increased astrocyte populations, increased astrocyte size, or some combinations of all of the above. (Papadimitriou et al., 2010.) In vivo diagnostic techniques largely focus on symptomatic features of ALS, including electrophysiological evaluations (e.g., nerve conduction, needle electromyography, transcranial magnetic stimulation), neuroimaging muscle biopsy, among others. However, each of these methods only measure advanced stages of symptomatic ALS, thereby limiting efficacy of prognosis, diagnosis, and therapeutic options. Thus, identifying molecules playing a direct role in ALS pathogenesis would provide therapeutic targets, improve understanding of the role of different cellular actors, while also increasing opportunities for improved detection, prognosis and diagnosis.

Described herein is a novel type of astrocyte-like cells displaying aberrant features, which resulted from isolation and characterization of astrocyte populations from the spinal cord of adult SOD1^{G93A} transgenic rats. An important feature of aberant astrocytes is production of toxic soluble factor(s) with a potent capacity to induce motor neuron death. These cells can be propagated in culture and are described as AbA cells (from Aberrant Astrocytes). Cultured AbA cells exhibit markers of undifferentiated astrocytes, increased proliferation, lack of replicative senescence, and marked toxic activity to cultured motor neurons. In the spinal cord of symptomatic rats, astrocytes displaying AbA cell markers were identified in close contact with motor neurons, providing physiological evidence for a pathogenic role in ALS disease progression.

As described, neurodegeneration in ALS is associated with activation and proliferation of glial cells surrounding damaged motor neurons. Astrocytes expressing ALS-linked SOD1 mutations specifically induce motor neurons loss and contribute to disease progression by yet unknown means. The present isolation of AbA cells from animals bearing the SOD1^{G93A} mutation now provides an accessible resource to characterize this unique astrocyte population with a view towards understanding the interactions causing the motor neuron loss that is a hallmark of ALS. For example, identifying toxic soluble factor(s) secreted by AbA cells may provide improved methods for evaluating ALS disease progression through detection and measurement of the soluble factor in a sample. Likewise, such factor(s) provide a potential therapeutic target to be inhibited in limiting or halting motor neuron death for ALS treatment.

Importantly, these cells can be maintained in culture during several passages without undergoing replicative senescence. AbA cells display multiple markers of undifferentiated astrocytes, including non-fibrillar GFAP, S100beta and connexin 43, but lack the GLT1 glutamate transporter and stem cell markers. Remarkably, AbA cells exhibit a 100-fold increased toxic activity to motor neurons in conditioned medium as compared to primary neonatal SOD1^{G93A} astrocytes. Further, AbA-like cells can be identified in the degenerating spinal cord as displaying a distinctive GFAP/S100b+ and Cx43/S100b+ immunoreactivity. Moreover, these ABA-like cells in the spinal cord also display active proliferation and close contact with motor neurons, suggesting an important pathological relevance. The emergence of aberrant astrocytes after disease onset may explain key aspects of ALS pathogenesis and defines a new diagnostic and therapeutic target for ALS and other neurodegenerative diseases.

Neuronal degeneration in ALS begins as a focal process that spreads contiguously through the upper and lower motoneuron levels. (Ravits J M, La Spada A R (2009) ALS motor phenotype heterogeneity, focality, and spread: deconstructing motor neuron degeneration. Neurology 73: 805-811.) This suggests an acquired pathogenic mechanism by which motoneuron pathology and inflammation actively propagate in the CNS. ABA-like cells serve as evidence for a specific population of spinal cord astrocytes that proliferate in the diseased ventral horn. In vitro, AbAs behave as neurotoxic cells through the secretion of factors with potent capacity for motoneuron killing. Thus, AbAs provide an important platform for unlocking biochemical pathways generating ALS conditions, while providing an immediate focus point for the role of astrocytes in in motor neuron degeneration and disease progression in ALS.

In addition to markers, toxicity and pathologically relevant localization, ABA cells possess a remarkably high proliferative potential that permits their isolation and oligoclonal expansion from primary adult spinal cord. Under identical conditions, age-matched non-transgenic rats yield few cells with much less growth potential. However, although the proliferation rate during the exponential growth phase is almost double that of the neonatal astrocytes, it is far below that of the C6 astrocyte cell line, discarding a fully transformed phenotype of AbAs. The fact that AbA cells do not follow replicative senescence may indicate altered function of growth-regulatory genes. Additionally, AbAs might represent a population of glial cells with undifferentiated features of precursor cells that have been reported to proliferate in ALS animal models. (Magnus T, et al. (2008) Adult glial precursor proliferation in mutant SOD1G93A mice. Glia 56: 200-208.) The generation of AbAs in the spinal cord seems to be associated to the overt inflammatory microenvironment that accompanies motoneuron loss, as particularly notable in SOD1^{G93A} rats. (Xie Y, et al. (2004) Inflammatory mediators and growth factors in the spinal cord of G93A SOD1 rats. Neuroreport 15: 2513-2516.) The unique inflammatory milieu of degenerating ALS spinal cord may promote the recruitment and phenotypic transition of glial cells or precursors along a specific pathway leading to AbAs. (Lepore AC, et al. (2008) Focal transplantation-based astrocyte replacement is neuroprotective in a model of motor neuron disease. Nat Neurosci 11: 1294-1301; Buffo A, et al. (2008) Origin and progeny of reactive gliosis: A source of multipotent cells in the injured brain. Proc Natl Acad Sci 105: 3581-3586.) Thus, AbAs represent an intriguing target for further understanding the pathogenesis of ALS and other neurodegenerative diseases.

Although AbA cultures are almost morphologically indistinguishable from primary neonatal astrocytes, the cells exhibit a set of distinctive antigenic astrocytic markers. Cultured AbAs express increased levels of vimentin and NG2, two markers suggestive of an immature phenotype. However, AbAs do not express markers of glial precursors, such as A2B5, and lack the potential of generating other types of glia cells following serum deprivation. Cultured AbA cells are characterized by high S100b and Cx43 expression and low levels of nonfilamentous GFAP. As a prototypic subunit of the Ca binding S100 proteins, S100b is known to exert paracrine effects in astrocytes and neurons. (Shiraishi N, Nishikimi M (1998) Suppression of copper-induced cellular damage by copper sequestration with S100b protein. Arch Biochem Biophys 357: 225-230; Hu J, et al. (1997) S100beta induces neuronal cell death through nitric oxide release from astrocytes. J Neurochem 69: 2294-2301.) Other roles include and contributes to astrocyte proliferation, migration, differentiation and neurotoxicity. (Brozzi F, et al. (2009) S100B Protein Regulates Astrocyte Shape and Migration via Interaction with Src Kinase: implications for astrocyte development, activation and tumor growth. J Biol Chem 284: 8797-8811; Donato R, et al. (2009) S100B's double life: intracellular regulator and extracellular signal. Biochim Biophys Acta 1793: 1008-1022; Raponi E, et al. (2007) S100B expression defines a state in which GFAP-expressing cells lose their neural stem cell potential and acquire a more mature developmental stage. Glia 55: 165-177; Hu J, Van Eldik L J (1996) S100 beta induces apoptotic cell death in cultured astrocytes via a nitric oxide-dependent pathway. Biochim Biophys Acta 1313: 239-245.) The fact that S100 proteins strongly bind Zn²⁺ ions suggests their overexpression could lead to the depletion of zinc in astrocytes interfering with the SOD1 metalation, a condition leading to aberrant oxidative chemistry of mutant SOD1. (Estevez A G, et al. (1999) Induction of nitric oxide-dependent apoptosis in motor neurons by zinc deficient superoxide dismutase. Science 286: 2498-2500; Beckman J S, et al. (2001) Superoxide dismutase and the death of motoneurons in ALS. Trends Neurosci 24: S15-20.) Intracellular S100b can interact with GAFP monomers to prevent assembly into glial filaments. Frizzo J K, et al. (2004) S100B-mediated inhibition of the phosphorylation of GFAP is prevented by TRTK-12. Neurochem Res 29: 735-740.) This may explain the diffuse GFAP distribution found in AbA cells. Remarkably, AbAs expressed several times more Cx43 than neonatal astrocytes. (Frizzo J K, et al. (2004) S100B-mediated inhibition of the phosphorylation of GFAP is prevented by TRTK-12. Neurochem Res 29: 735-740.) Since Cx43 modulates astrocyte proliferation, migration and differentiation, the functional significance of high Cx43 expression in AbA is currently under study. In inflammatory astrocytes, Cx43 can form either intercellular gap junctions or hemichannels that have been linked to neurodegeneration. (Homkajorn B, et al. (2010) Connexin 43 regulates astrocytic migration and proliferation in response to injury. Neurosci Lett; Orellana J A, et al. (2009) Modulation of brain hemichannels and gap junction channels by pro-inflammatory agents and their possible role in neurodegeneration. Antioxid Redox Signal 11: 369-399.) Finally, the blunted levels of astroglial glutamate transporter (GLT1) protein in AbAs suggests these cells recapitulate a defect in GLT1 expression previously described in ALS patients and SOD1^{G93A} rats. (Jackson M, et al. (1999) Polymorphisms in the glutamate transporter gene EAAT2 in European ALS patients. J Neurol 246: 1140-1144; Meyer T, et al. (1999) The RNA of the glutamate transporter EAAT2 is variably spliced in amyotrophic lateral sclerosis and normal individuals. J Neurol Sci 170: 45-50; Lin C L, et al. (1998) Aberrant RNA processing in a neurodegenerative disease: the cause for absent EAAT2, a glutamate transporter, in amyotrophic lateral sclerosis. Neuron 20: 589-602; Howland D S, et al. (2002) Focal loss of the glutamate transporter EAAT2 in a transgenic rat model of SOD1 mutant-mediated amyotrophic lateral sclerosis (ALS). Proc Natl Acad Sci 99: 1604-1609.) Since GLT1 is expressed in differentiated astrocytes endings to prevent glutamate-induced excitotoxicity of motoneurons, accumulation of AbAs around motoneurons may further promote excitotoxic damage of motoneurons in vivo.

Previous studies have shown that astrocytes bearing the SOD1^{G93A} mutation induce apoptosis of embryonic or stem cell-derived motor neurons in both coculture conditions and through soluble factors found in the culture media. (Vargas et al. 2006, Di Giorgio et al., 2007; Nagai et al., 2007; Yamanaka K, et al. (2008) Mutant SOD1 in cell types other than motor neurons and oligodendrocytes accelerates onset of disease in ALS mice. Proc Natl Acad Sci 105: 7594-7599; Gong Y H, et al. (2000). Restricted expression of G86R Cu/Zn superoxide dismutase in astrocytes results in astrocytosis but does not cause motoneuron degeneration. J Neurosci 20: 660-665.) The inventors show herein that AbAs display an unprecedented toxicity to motoneurons that vastly surpasses that of neonatal transgenic astrocytes. Remarkably, motor neurons failed to survive when plated on a confluent layer of AbA cells and AbA conditioned medium is >10-fold more potent in inducing motor neuron death than neonatal Tg astrocytes. The potency of ABA conditioned media suggests a mechanism of AbA toxicity to motoneurons acting through secreted factors may act with local activity or migratory spreading locally or migratory spread of the disease through diffusion to the cerebrospinal fluid.

Supporting the view that AbA cells play a vital role in pathogenesis of ALS, similar ABA-like cells are found in the degenerating spinal cord, express both Cx43 and S100B and thereby indicate a common phenotype with those AbA cells isolated in culture. Little is known about the significance of Cx43-overexpressing astrocytes in ALS. However, previous evidence indicates that neurogenic activation of astrocytes is sufficient to induce Cx43 in spinal cord astrocytes following axotomy of facial nerve and spinal cord injury, which suggests a pathogenic role of astrocytic CX43 upregulation. (Rohlmann A, et al. (1993) Facial nerve lesions lead to increased immunostaining of the astrocytic gap junction protein (connexin 43) in the corresponding facial nucleus of rats. Neurosci Lett 154: 206-208; Lee IH, et al. (2005) Glial and neuronal connexin expression patterns in the rat spinal cord during development and following injury. J Comp Neurol 489: 1-10; Migheli A, et al. (1999) S-100beta protein is upregulated in astrocytes and motor neurons in the spinal cord of patients with amyotrophic lateral sclerosis. Neurosci Lett 261: 25-28.) AbA-like cells were in close contact with degenerating motoneurons and this observation is in agreement with previous studies in ALS patients showing S100b-positive astrocytes being localized in the anterior horns, in close contact with motoneurons. (Migheli et al., 1999) Additionally, S100b forms heterodimers with other members of S100 proteins including S100A6. (Yang Q, et al. (1998) Demonstration of heterodimer formation between S100B and S100A6 in the Yeast Two-Hybrid system and human melanoma. Experimental Cell Research 246: 501-509; Deloulme J C, et al. (2000) S100A6 and S100A11 are specific targets of the calcium- and zinc-binding S100B protein in vivo. J Biol Chem 275: 35302-35310.) Further, S100B is specifically upregulated in reactive astrocytes occurring in ALS patients and mutant SOD1 transgenic animals. (Hoyaux D, et al. (2002) S100A6 overexpression within astrocytes associated with impaired axons from both ALS mouse model and human patients. J Neuropathol Exp Neurol 61: 736-744; Hoyaux D, et al. (2000) S100A6, a calcium- and zinc-binding protein, is overexpressed in SOD1 mutant mice, a model for amyotrophic lateral sclerosis. Biochim Biophys Acta 1498: 264-272.) Together, these results indicate the possible emergence of aberrant astrocytes in ALS patients.

Thus, a key question on ALS pathogenesis is addressed herein by the identification and isolation of a previously unknown astrocyte population with the potential to mediate progressive motoneuron disease and inflammation. Isolation and expansion of AbA cells provides a valuable model for evaluating pathways involved in ALS pathogenesis and for identifying targets for diagnosis and therapies slowing ALS progression.

Various embodiments of the present invention are based, at least in part, upon theses findings.

The present invention provides for an isolated population of neurotoxic astrocytes, also referred to herein as aberrant astrocytes (“AbA cells”). In one embodiment, the isolated population of neurotoxic astrocytes is obtained from a mammal expressing the superoxide dismutase1 G93A (SOD1^{G93A}) mutation. In a particular embodiment, the mammal is a transgenic rat expressing the SOD1^{G93A} mutation. In another particular embodiment, the mammal is a symptomatic transgenic rat expressing the SOD1^{G93A} mutation.

In one embodiment, the isolated population of aberrant astrocytes displays markers of undifferentiated astrocytes. In various embodiments, the isolated population of aberrant astrocyte expresses at least one of the markers selected from the group consisting of GFAP, nestin, vimentin, S100β, CX43, GLAST, GLT1, glutamine synthase, Ng2, Olig2, A2B5, ED1, and combinations thereof. In various embodiments, the markers of undifferentiated astrocytes are selected from the group consisting of non-fibrillar GFAP, S100beta, connexin 43 and combinations thereof. In one particular embodiment, isolated population of aberrant astrocytes displays the following markers: non-fibrillar GFAP, S100beta, and connexin 43. In another embodiment, the isolated population of aberrant astrocytes lacks the GLT1 glutamate transporter and stem cell markers. In another embodiment, the isolated population of aberrant astrocytes displays GFAP/S100b+ and Cx43/S100b+ immunoreactivity. In another embodiment, the isolated population of aberrant astrocytes display active proliferation and are in close contact with motor neurons. In another embodiment, the isolated population of aberrant astrocytes exhibits increased toxic activity to motor neurons in conditioned medium compared to primary neonatal SOD1^{G93A} astrocytes or normal astrocytes. In a particular embodiment, the isolated population of aberrant astrocytes exhibits at least 100-fold increased toxic activity to motor neurons in conditioned medium compared to primary neonatal SOD1^{G93A} astrocytes.

The present invention also provides for a method of isolating the aberrant astrocytes. The method comprises providing a population of cells comprising an aberrant astrocyte; placing a layer of agarose over the population of cells; culturing the population of cells under the layer of agarose to allow proliferation of the aberrant astrocytes; separating the agarose layer from the population of cells; and plating/culturing the population of cells to allow proliferation of the aberrant astrocytes. In another embodiment, the method further comprises selecting the aberrant astrocytes. In one embodiment, the population of cells is obtained from the spinal cord of a mammal. In another particular embodiment, the population of cells is obtained from a degenerating spinal cord of a mammal. In a particular embodiment, the mammal is a transgenic rat expressing the SOD1^{G93A} mutation. In another particular embodiment, the mammal is a symptomatic transgenic rat expressing the SOD1^{G93A} mutation. In another embodiment, the mammal is a human subject with ALS. In another embodiment, the population of cells is obtained from autopsy tissue from human ALS patients; for example, motor cortex, brain stem, and/or spinal cord.

The present invention also provides for a method to diagnose a neurodegenerative disease. In one embodiment, the neurodegenerative disease is selected from the group consisting of amyotrophic lateral sclerosis (ALS), motor neuron disease, spinal cord injury, Parkinson's disease, Alzheimer's disease, Huntington's disease and combinations thereof. In one embodiment, the neurodegenerative disease involves reactive astrocytosis. In one particular embodiment, the neurodegenerative disease is amyotrophic lateral sclerosis (ALS). In one embodiment, the method comprises detecting an aberrant astrocyte; and determining the presence of the neurodegenerative disease when the presence of the aberrant astrocyte is detected. In one embodiment, the aberrant astrocyte is detected from a tissue sample. In another embodiment, the aberrant astrocyte is detected from the motor cortex, brain stem and/or spinal cord.

In various embodiments, the aberrant astrocyte that is detected displays a marker of an undifferentiated astrocyte. In various embodiments, the aberrant astrocyte expresses at least one of the markers selected from the group consisting of GFAP, nestin, vimentin, S100β, CX43, GLAST, GLT1, glutamine synthase, Ng2, Olig2, A2B5, ED1, and combinations thereof. In one embodiment, the marker of the undifferentiated astrocyte is selected from the group consisting of non-fibrillar GFAP, S100beta, connexin 43, nestin, vimentin, Ng2, A2B5 and combinations thereof. In one particular embodiment, the aberrant astrocyte displays the following markers: non-fibrillar GFAP, S100beta, and connexin 43. In another embodiment, the aberrant astrocyte lacks the GLT1 glutamate transporter and stem cell markers. In another embodiment, the aberrant astrocyte displays GFAP/S100b+ and Cx43/S100b+ immunoreactivity. In another embodiment, the aberrant astrocyte displays active proliferation and is in close contact with motor neurons. In another embodiment, the aberrant astrocyte exhibits increased toxic activity to motor neurons in conditioned medium compared to primary neonatal SOD1^{G93A} astrocytes. In a particular embodiment, the aberrant astrocyte exhibits at least 100-fold increased toxic activity to motor neurons in conditioned medium compared to primary neonatal SOD1^{G93A} astrocytes.

Another embodiment provides for a method to diagnose a neurodegenerative disease by detecting the presence of secretomes (secreted protein) of an aberrant astrocyte. In one embodiment, the method comprises providing a biological sample from a subject; detecting in the biological sample the presence of one or more secretomes listed in Table 2, Table 3, or both Tables 2 and 3; and determining the presence of the neurodegenerative disease when the presence of one or more secretomes are detected. In a particular embodiment, the one or more secretomes are from Table 2. In another particular embodiment, the one or more secretome are from Table 3.

In various embodiments, a method of detecting a neurodegenerative disease can be established by obtaining an experimental sample from a subject suspected of having a neurodegenerative disease, measuring the level of a protein produced by an aberant astrocyte in the experimental sample, comparing the level of the protein from the experimental sample to the level of the protein in a subject without the neurodegenerative disease as a control sample, wherein a difference in the level of the protein detects the neurodegenerative disease. In various embodiments, measurements from a control sample can be the same or different cell or tissue types as that of the experimental sample, various cell lines, obtained from the subject suspected of having a neurodegenerative disease, a single healthy (i.e., non-diseased) subject, a population of healthy subjects, and/or subjects with varying degress of disease progression. Differences in measured parameters, as between experimental and control samples, may be statistically significant differences according to various techniques known in the art, including difference one- or two-sample z-tests, t-tests, chi-square tests, and f-tests with pooled or proportional variations of these tests. Different distributions such as Gaussian distribution, student's t-distribution may be used, with p-values such as 10%, 5% 1% and/or 0.1% being common methods of assessing statistical significance.

Examples of biological samples include but are not limited to mammalian body fluids, sera such as blood (including whole blood as well as its plasma and serum), cerebrospinal fluid (CSF), urine, sweat, saliva, tears, pulmonary secretions, breast aspirate, prostate fluid, seminal fluid, stool, cervical scraping, cysts, amniotic fluid, intraocular fluid, mucous, moisture in breath, animal tissue, cell lysates, tumor tissue, hair, skin, buccal scrapings, nails, bone marrow, cartilage, prions, bone powder, ear wax, etc. or even from external or archived sources such as tumor samples (i.e., fresh, frozen or paraffin-embedded). Samples, such as body fluids, sera, or CSF obtained during the course of clinical trials may be particularly advantageous for use in connection with the present invention, although samples obtained directly from living subjects under alternate conditions or for other purposes may be readily used as well. In one particular embodiment, the biological sample is CSF. In another embodiment, the biological sample is from a human subject with ALS. In another embodiment, biological sample is autopsy tissue from human ALS patients; for example, motor cortex, brain stem and/or spinal cord. In another embodiment, biological sample is tissue from human ALS patients; for example, motor cortex, brain stem spinal cord and/or. skeletal muscle. In various embodiments, diagnostic procedures may be based on immune-based ELISA detection, mass spectrometry or electrophoresis of the proteins specifically secreted by aberrant cells, but not by normal astrocytes or glial cells. In another embodiment, using a single or combined set of binders to aberrant astrocyte cells could be applied to neuroimaging techniques for determining disease progression.

The present invention also provides a method for treatment for a neurodegenerative disease. In one embodiment, the neurodegenerative disease is selected from the group consisting of amyotrophic lateral sclerosis (ALS), motor neuron disease, spinal cord injury, Parkinson's disease, Alzheimer's disease, Huntington's disease and combinations thereof. In one embodiment, the neurodegenerative disease involves reactive astrocytosis. In one particular embodiment, the neurodegenerative disease is amyotrophic lateral sclerosis (ALS). In one embodiment, the method comprises selecting a subject in need of treatment for a neurodegenerative disease, administering a compound that modulates the activity of an aberrant astrocyte, wherein the modulation of the activity of an aberrant astrocyte treats the subject. In one embodiment, the compound directly targets antigens or ligands associated with an aberrant astrocyte to modulate the activity of the aberrant astrocyte. In another embodiment, the compound targets a trophic, metabolic or signaling pathway associated with aberrant astrocyte activity. In another embodiment, the compound modulates inflammation and disease progression associated with aberrant astrocyte activity. In various embodiments, the compound modulates a pathway related to one or more secretomes listed in Table 2, Table 3, or both Tables 2 and 3. In a particular embodiment, the one or more secretomes are from Table 2. In another particular embodiment, the one or more secretome are from Table 3. In various embodiments, compounds may be nucleic acids, antibodies, proteins such as soluble receptors, and small molecules,

The present invention also provides for a method of screening for drug that may treat a neurodegenerative disease or slow the progressing of the neurodegenerative disease. One embodiment provides for a method of identifying a compound that inhibits the proliferation of the aberrant astrocytes comprising: providing an isolated population of aberrant astrocytes; contacting the isolated population of aberrant astrocytes with a test compound; and determining whether the test compound inhibits the proliferation of the aberrant astrocytes, wherein a decreased proliferation of the aberrant astrocytes is an indication that the test compound is inhibiting the proliferation of the aberrant astrocytes. In one embodiment, the decreased proliferation of the aberrant astrocytes is determined by comparison to a control experiment or a standardized proliferation level.

Another embodiment provides for a method of identifying a compound that is cytotoxic to the aberrant astrocytes comprising: providing an isolated population of aberrant astrocytes; contacting the isolated population of aberrant astrocytes with a test compound; and determining whether the test compound is cytotoxic to the aberrant astrocytes, wherein a decrease in the number of live aberrant astrocytes in the isolated population of aberrant astrocytes is an indication that the test compound is cytotoxic to the aberrant astrocytes.

Another embodiment provides for a method of identifying a compound that inhibits a cytotoxic secretome from aberrant astrocytes comprising: providing an isolated population of aberrant astrocytes; contacting the isolated population of aberrant astrocytes with a test compound; and determining whether the test compound is inhibits motor neuron death, wherein a decrease in the number of motor neuron cell death is an indication that the test compound inhibits the cytotoxic secretome from the aberrant astrocytes. In various embodiments the secretome is selected from Table 2 and Table 3. In one embodiment, the decrease in the number of motor neuron cell death is determined by comparison to a control experiment or a standardized motor neuron cell death level.

In various embodiments, methods of identifying compounds capable of modulating aberrant astrocyte activity can be established by measuring a parameter of aberrant astrocytes such as proliferation, cytotoxicity, alterations in a protein function, or alterations in a protein expression, and combinations thereof, following contact with a test compound and comparing this experimental sample to the same parameter measured from a population of aberrant astrocytes not contacted with the test compound as a control sample, wherein a difference in the measured parameter, identifies the compound as capable of modulating aberrant astrocyte activity. In various embodiments, measurements from a control sample population can be drawn from the same or different cell or tissue type as that of the experimental sample, various cell lines, obtained from the subject suspected of having a neurodegenerative disease, a single healthy (i.e., non-diseased) subject, a population of healthy subjects and/or subjects with varying degress of disease progression. Differences in measured parameters, as between experimental and control samples, may be statistically significant differences according to various techniques known in the art, including difference one- or two-sample z-tests, t-tests, chi-square tests, and f-tests with pooled or proportional variations of these tests. Different distributions such as Gaussian distribution, student's t-distribution may be used, with p-values such as 10%, 5% 1% and/or 0.1% being common methods of assessing statistical significance.

The methods of identifying compounds that inhibit the proliferation of the aberrant astrocytes, compounds that are cytotoxic to the aberrant astrocytes or compounds that inhibit a cytotoxic secretome from aberrant astrocytes may be performed as high-throughput screening (“HTS”) assays. Through a combination of robotics, data processing and control software, liquid handling devices, and/or sensitive detectors, HTS may allow for a vast number of biochemical, genetic or pharmacological tests to be conducted in a short period of time. Through HTS, one can rapidly identify active compounds, antibodies or genes which modulate a particular biomolecular pathway. In one embodiment, HTS may be used to screen for compounds that may inhibit the proliferation of the aberrant astrocytes. In another embodiment, HTS may be used to screen for compounds that may be cytotoxic to the aberrant astrocytes. In another embodiment, the HTS may be used to screen for compounds that may inhibit cytotoxic secretomes from aberrant astrocytes. The results of HTS may provide starting points for drug design and for understanding the interaction or role of a particular biochemical process in biology; for example, motor neuron disease.

HTS is an approach to collect a large amount of experimental data in a relatively short time. A HTS may utilize a plate (e.g., a small container with wells). The wells may contain a different test compound for each well and/or an empty well for use as a control. An assay may be performed by filling each well of the plate with the cells of the present invention. After a period of incubation to allow the cells to react or fail to react with the compounds in the wells, measurements may be taken across all the plate's wells, either manually or by a machine. A specialized automated analysis machine may be used to detect the fluorescence from the cells. One skilled in the art will recognize other HTS methods that may utilize the inventive cells.

The test compound used in the inventive method may be any compound known in the art. Examples of compound libraries that may be used include but are not limited to the ChemBridge DiverSet library (approximately 30,000 chemically diverse small molecules).

The present invention is also directed to kits to diagnose a neurodegenerative disease, to screen for compounds that are useful for inhibiting the proliferation of aberrant astrocytes, compounds that are cytotoxic to the aberrant astrocytes, or compounds that inhibit cytotoxic secretomes from aberrant astrocytes. The kit is useful for practicing the aforementioned inventive methods. The kit is an assemblage of materials or components, including at least one of the inventive compositions. Thus, in some embodiments the kit contains a population of aberrant astrocytes as described above.

The exact nature of the components configured in the inventive kit depends on its intended purpose. For example, some embodiments are configured for the purpose of diagnosing a neurodegenerative disease, to screen for compounds that are useful for inhibiting the proliferation of aberrant astrocytes, compounds that are cytotoxic to the aberrant astrocytes, or compounds that inhibit cytotoxic secretomes from aberrant astrocytes. In one embodiment, the kit is configured particularly for the purpose of diagnosing mammalian subjects. In another embodiment, the kit is configured particularly for the purpose of diagnosing human subjects. In further embodiments, the kit is configured for veterinary applications, diagnosing subjects such as, but not limited to, farm animals, domestic animals, and laboratory animals.

Instructions for use may be included in the kit. “Instructions for use” typically include a tangible expression describing the technique to be employed in using the components of the kit to effect a desired outcome. Optionally, the kit also contains other useful components, such as, diluents, buffers, culture media, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a vial used to contain suitable quantities of isolated aberrant astrocytes. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Materials

Media and chemicals for cell culture (Dulbecco's modified Eagle's medium, Neurobasal and L15 media, horse and fetal bovine serum, penicillin/streptomycin, trypsin, polylysine, laminin) were from Invitrogen. Commercial antibodies were from Dako (Carpinteria, Calif., USA), Abcam (Cambridge, Mass., USA), Sigma (St Louis, Mo., USA), Covance (Princeton, N.J., USA), Cell Signaling (Danvers, Mass., USA) and Invitrogen. All other chemicals of analytical grade were obtained from Sigma. Reagents used in Western blotting were from Pierce (IL, USA).

### Example 2

### Animals

All procedures using laboratory animals were in accordance with the international guidelines for the use of live animals and were approved by the Institutional Animal Committee. Male hemizygous NTac:SDTgN(SOD1^{G93A})L26H rats (Taconic, Hudson, N.Y.), originally developed by Howland et al., were bred locally by crossing with wild-type female rats. Male SOD1^{G93A} progenies were used for further breeding to maintain the line. The breeding and experimental protocols were approved by the Institutional Animal Use Committee. Rats were housed in a centralized Animal Facility with a 12-hr light-dark cycle with ad libitum access to food and water. SOD1^{G93A} progenies were verified by PCR genotyping according to Howland 2002. Symptomatic disease onset was determined by periodic clinical examination of abnormal gait, typically expressed as a subtle limping or dragging of one hindlimb. Rats were euthanized when reaching the endstage of the disease. Finally, the symptomatic disease onset (163 days) and lifespan (180 days) in this colony were considerably delayed compared to what were earlier described. (Howland et al., 2002.)

### Example 3

### Isolation of AbA Cells from Animals

AbA cells were obtained from adult spinal cord of transgenic symptomatic SOD1^{G93A} rats (175 days) according to the procedures described by Saneto and De Vellis with minor modifications. (Saneto R P and J De Vellis (1987) Neuronal and glial cells: Cell culture of the central nervous system. In: Turner, A. J.; Bachelard, H. S., eds. Neurochemistry: a practical approach (IRL. Press, Oxford-Washington, D.C.), pp 27-63; Cassina P et al. (2002) Peroxynitrite triggers a phenotypic transformation in spinal cord astrocytes that induces motor neuron apoptosis. J Neurosci Res 67:21-29.) Adult aged-matched non-transgenic rats were used as controls. Briefly, animals were sacrificed by deep anesthesia and spinal cord was dissected on ice. After removing the meanings, spinal cord was chopped and dissociated with trypsin and mechanically. The resulting extract was passed through a 80 mm mesh in order to eliminate tissue debris and then spun. Pellet was resuspended in culture media and then plated in a 25 cm² tissue culture flask. Since large amounts of fat debris hindered cell counting, the method was standardized to plate the cells isolated from one spinal cord in one 25 cm² tissue culture flask. After 20 days in bottle, cells were replated every week

### Example 4

### Agarose Macromolecular Trap for Enhancing Autocrine and Paracrine Transformative Effect on AbA Cell Cultures

An “agarose macromolecular trap” method can be used to isolate AbA cells from the degenerating spinal cord of rats expressing the SOD1^{G93A} mutation.

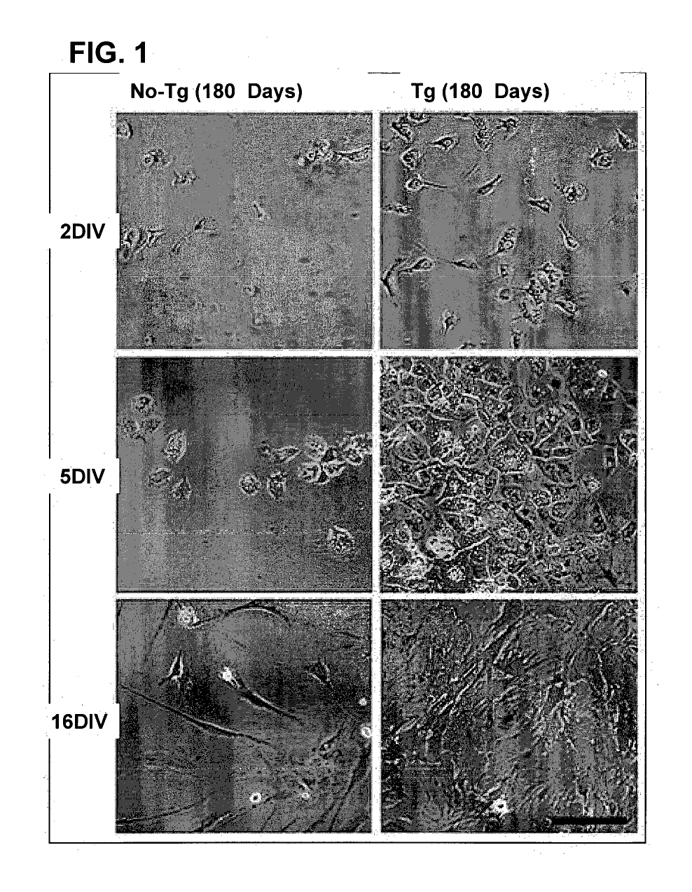

Primary cultures were established from the spinal cord of asymptomatic and symptomatic SOD1^{G93A} rats (Tg-A, Tg-S) and compared to those from non-transgenic (NTg) littermates of the same age (FIG. 1). The cultures from NTg and Tg-A rats (180 days old) yielded a few amount of cells attached in DIV 2 that slowly proliferated in the following days, but failed to reach confluence or resisted subsequent passages. In contrast, primary cultures from symptomatic rats (170-180 days old) yielded a large number of cells, most of them displaying multiple cytoplasmic vacuoles resembling macrophagic microglia (FIG. 1). A population of cells rapidly proliferated by DIV 5 from apparent oligomeric clusters, but isolated cells were also seen.

At this time, a 3-4 mm layer of agarose maintained at 37° C. was gently added to the culture flask and let to polymerize on the top of the cell layer. While agarose layer did not attach to the cell layer, it formed a trap for macromolecules released by inflammation (such as proteins and glycoproteins), allowing them to concentrate to critical levels nearby the cell layer. The inventors found that the agarose layer potently stimulated cell proliferation by autocrine and paracrine mechanisms in Tg-S cultures but had no effect in asymptomatic rats. The agarose layer mediates a “transforming effect” (increased proliferation, lack of replicative senescence, undifferentiated features), when glial cells come from a degenerating spinal cord. The source of the transforming factor is likely the activated glial cells from the symptomatic animals and not those arising from healthy ones. The molecular trap with agarose facilitates the autocrine and paracrine effects of factors that are secreted to the medium by the inflammatory cells, leading to subsequent transformation of precursors cells. FIG. 1 shows that after 16 days (DIV 16) under the agarose trap the Tg-S cultures formed a confluent monolayer of flat cells with numerous superficial cells displaying processes, as compared to only few viable cells in NTg or Tg-A.

After withdrawal of the agarose layer and subsequent replating, Tg-S cells vigorously proliferated to yield highly homogeneous monolayers of packed flat, fusiform to polygonal cells resembling astrocytes (FIG. 1). These cells were successfully passaged and propagated during 10 months (corresponding to 30-35 passages) without undergo replicative senescence. Beyond passage 20, cells increased the proliferation rate and underwent phenotypic transformation.

Because of the unique proliferative behavior and phenotypic features, the inventors referred to these cells as AbAs (Aberrant Astrocytes). AbAs were obtained in 100% of cultures prepared from Tg-S rats, suggesting that they represent a common cell type associated to the symptomatic phase in rats undergoing motor neuron disease. Vacuolated AbA-like cells could be also obtained from the spinal cord of symptomatic SOD1^{G93A} mice, but the cells divided more slowly and failed to survive for successive passages.

### Example 5

### General Features and Initial Characterization of ABA Cells in Culture

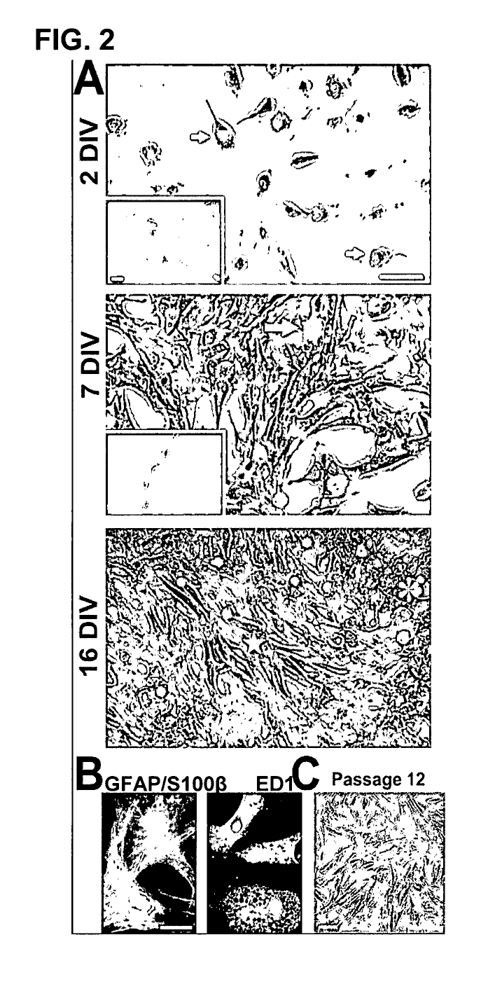

ABA cells isolated from degenerating spinal cord of symptomatic adult transgenic SOD1^{G93A} (Tg-Symp, 170 days old) were compared to asymptomatic SOD1^{G93A} (Tg-Asymp, 90 days old) and non-transgenic rats (non-Tg, 170 days old). As before, cultures from Tg-Asymp or non-Tg rats yielded comparable pictures of low amount of cells in DIV2 (2 days in vitro), that slowly proliferated in the following days, but failed to reach confluence or resist subsequent passages (inserts in FIG. 2A). In contrast, cultures from Tg-Symp rats yielded a large number of cells that rapidly proliferated by DIV7, forming clusters of elongated flat cells resembling astrocytes associated to numerous phagocytic microglia (FIG. 2A, white arrows). By DIV16-20, the population with astrocytic phenotype increased sharply and organized as a monolayer bearing sparse microglial cells (FIG. 2A). At this time, astrocyte-like cells displayed intense immunoreactivity to S100b and CX43 but weak labeling to GFAP, while microglia were strongly labeled with ED1 (FIG. 2B). After subsequent replating, the cultures became devoid of microglia while astrocytes vigorously proliferated to yield highly homogeneous monolayers of flat, fusiform to polygonal cells (FIG. 2C). These cells were successfully passaged and propagated during the course of over 1 year without undergoing replicative senescence. However, after passage 20, cells underwent a phenotypic transformation, increasing the proliferation rate and losing the typical astrocyte morphology. AbA cells were generated systematically from several cultures prepared from symptomatic transgenic rats, suggesting they represent a common cell type resident in the degenerating spinal cord during the symptomatic phase of the disease.

### Example 6

### AbA Cell Proliferation

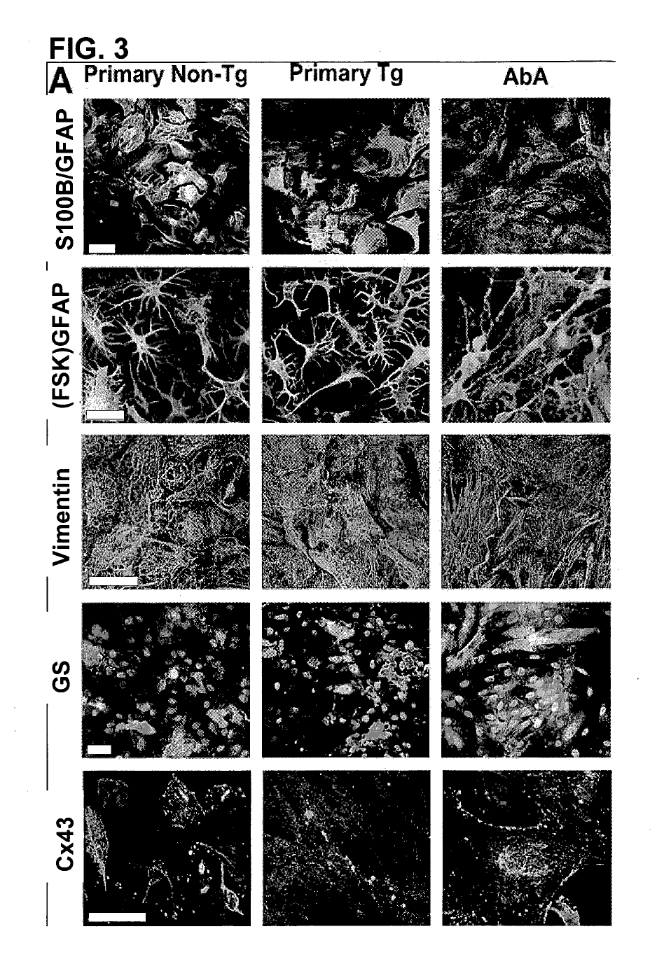

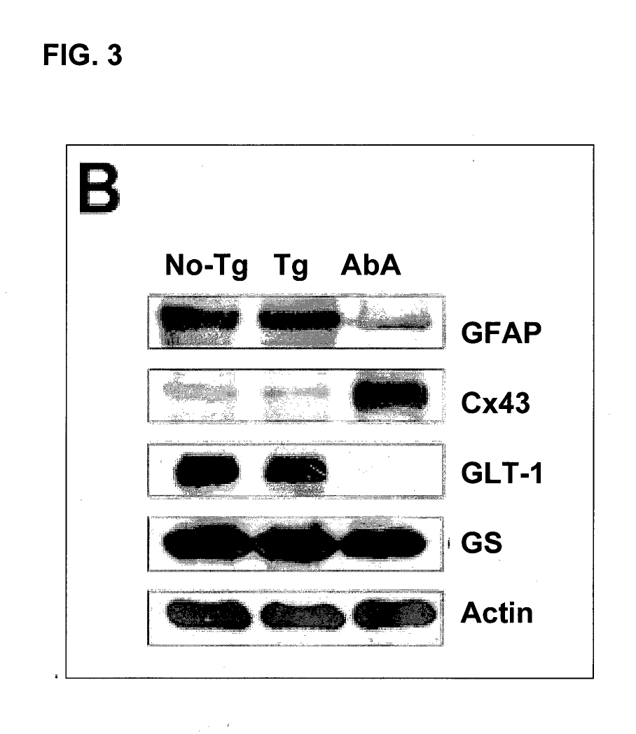

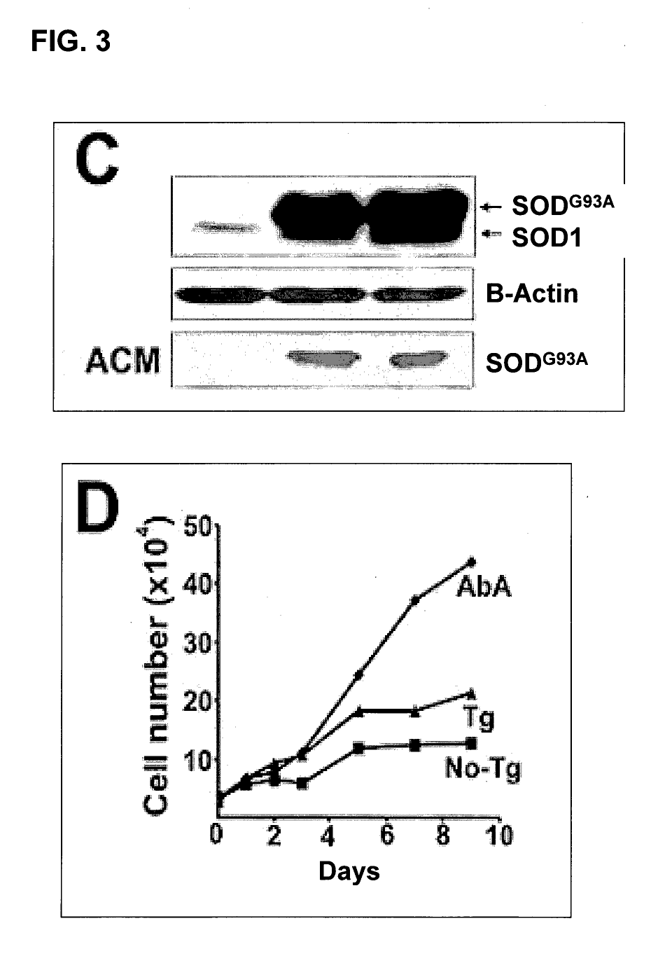

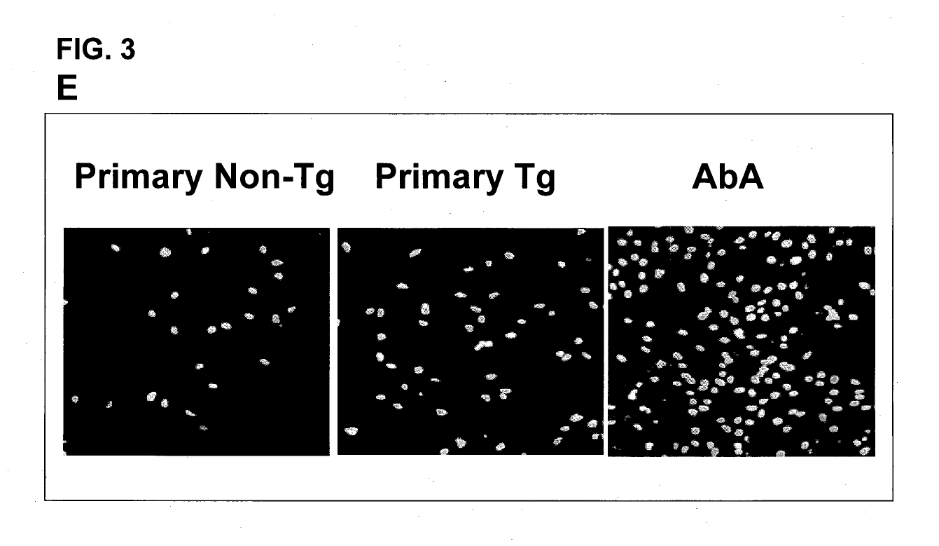

AbA cells or primary neonatal astrocytes were plated in 35 mm dishes at 12×103 cells/cm2. Six hours after plating, cells were harvested and counted to obtain the cell number at day zero. Counting occurred for 9 days as indicated in the FIG. 3D. Cells were lysed and intact nuclei were counted with a hemacytometer as described previously. (Ilieva H, et al. (2009) Non-cell autonomous toxicity in neurodegenerative disorders: ALS and beyond. J Cell Biol 187: 761-772; Hall ED, et al. (1998) Relationship of microglial and astrocytic activation to disease onset and progression in a transgenic model of familial ALS. Glia 23: 249-256.) Only healthy nuclei with clearly defined limiting membranes and phase bright were considered for quantification. The counts were performed in duplicate.

FIG. 3D shows the growth kinetics of AbA cells at passage 10 in media supplemented with 10% FBS, as compared to primary neonatal astrocytes. The growth rate of AbA cells was higher than transgenic and non-transgenic neonatal astrocytes but approximately 8 times lower than C6. The doubling times, calculated during the linear phase of the growth curve, was 30 h for AbA cells, 52 h for non-transgenic astrocytes and 49 h for transgenic astrocytes. As depicted in the growth curve (FIG. 3D), the AbA cell number continued to increase after reaching confluence at DIV5, suggesting a defective contact inhibition.

### Example 7

### Western Blot Analysis

For cellular proteins extraction, astrocytes and AbA monolayers were washed with PBS and whole cell extracts prepared in lysis buffer (50 mM HEPES pH 7.5, 50 mM NaCl, 1% Triton X-100, and complete protease inhibitor mixture, (Sigma), and then sonicated 3 times for 3 sec. Protein concentration was measured with a Bicinchoninic Acid (BCA) kit (Sigma). Then, protein extracts were placed in loading buffer containing 15% SDS, 0.3 M Tris pH 6.8, 25% Glycerol, 1.5 M β-mercaptoethanol and 0.01% Bromphenol Blue. Protein samples (40 μg) were resolved on 12% SDS polyacrylamide gel and transferred to PVDF membrane (Amersham, N.J., USA). Membranes were blocked for 1 h in Tris-buffered saline (TBS), 0.1% Tween-20 and 5% nonfat dry milk, followed by an overnight incubation with the correspondent primary antibody diluted in the same buffer. After washing with 0.1% Tween in TBS, the membrane was incubated with peroxidase-conjugated secondary antibody (Biorad, Calif., USA) for 1 h, washed and developed using the ECL chemiluminescent detection system (Amersham, N.J., USA). Primary antibodies were used at dilutions recommended by the manufacturers: GFAP (Sigma G9269, 1:4000), Cx43 (Polyclonal antibody from Berhoud et al., 1:1000), S100β (Sigma S2532, 1:400), Vimentin (Dako M0725, 1:250), GLT1 (Cell Signaling 3838 1:1000), Glutamine synthase (Abcam ab49873, 1:5000), Ng2 (Milipore AB5320, 1:1000), Olig2 (Abcam ab 33427, 1:400), A2B5 (Abcam ab 53521, 1:400), ED1 (Abcam ab31630, 1:500), SOD1^{G93A} (Santa Cruz Fla.-164, 1:3000).

### Example 8

Immunocytochemistry

Cultured cells were fixed with absolute methanol at −20° C. during 5 minutes on ice and then washed 3 times with 10 mM phosphate buffer saline (PBS, pH 7.4). Blockade of non specific binding was done incubating fixed cells with 5% bovine serum albumin:PBS during 1 h at room temperature. Primary antibodies were diluted in blocking solution and incubated overnight at 4° C. in a wet closed chamber. After several washes and 2 h of incubation with correspondent secondary antibodies conjugated to fluorescent probes (1:1000, Invitrogen), cells were mounted and imaged in an Olympus FV300 laser scanning confocal microscope. Immunocytochemistry primary antibody dilutions were as follows: GFAP (Sigma G9269, 1:400), Cx43 (Polyclonal antibody from Berhoud et al., 1:500), S100β (Sigma S2532, 1:400), Vimentin (Dako M0725, 1:250), GLT1 (Cell Signaling 3838 1:1000), Glutamine synthase (Abcam ab49873, 1:500), Ng2 (Milipore AB5320, 1:250), Olig2 (Abcam ab 33427, 1:400), A2B5 (Abcam ab 53521, 1:400), ED1 (Abeam ab31630, 1:500), SOD1^{G93A} (Santa Cruz Fla.-164, 1:3000).

### Example 9

### Immunohistochemistry

Animals were deeply anesthetized and submitted to transcardially perfusion with 0.9% saline and 10% PFA (in 0.1M phosphate buffer saline (PBS), pH 7.2-7.4) at constant 1 ml/min flow. Fixed spinal cord was removed, post-fixed by immersion during 24 h and then serially transverse sectioned on a vibrating microtome at 30-50 μm. Serial sections were collected in 100 mM PBS for immunohistochemistry. Free-floating sections were permeabilized 15 min at room temperature with 0.1% Triton X-100 in PBS, passed through washing buffered solutions, blocked with 5% BSA:PBS 30 min M at room temperature, and incubated overnight at 4° C. in a pH 7.4 0.1% Triton X-100 —PBS buffered solution containing the primary antibodies. Antibody detection was made by confocal microscopy using a confocal Olympus FV300 microscope. The same protocol was applied to recognize all of antigens studies except for BrdU and Ki67 in which antigen retrieval was performed according to manufacturer instructions.

### Example 10

### Antigenic Profile of AbA cells

The antigenic profile of AbA cell was characterized between passages 8-15. For comparison, the inventors used the well-characterized primary spinal cord astrocytes prepared from neonatal transgenic and non-transgenic rats. Since the inventors were unable grow and passage astrocytes from non-Tg adult rats, the phenotypic features of AbAs were compared with primary spinal cord astrocytes prepared from neonatal SOD1^{G93A} rats and non-transgenic littermates. This antigenic profile is listed in Table 1, wherein a single X refers to low intensity, XX refers to intermediate intensity, and XXX refers to high intensity with qualitative differences in GFAP staining indicated in (brackets).

Among the antigenic markers listed in Table 1, it was found that both AbAs and neonatal astrocytes expressed typical astrocytic markers such as GFAP, vimentin, S100b, Cx43, glutamate transporter (GLAST) and glutamine synthase, except glutamate transporter (GLT1) protein that was not detected in AbAs. In addition, about 80% of AbAs were labeled with antibodies to NG2 proteoglycan as compared to only 20-30% of primary neonatal astrocytes. AbA cells were negative for A2B5, Olig2 and ED1, which are expressed by glial precursors, oligodendrocyte progenitor cells and phagocytic microglia, respectively.

TABLE 1
Comparative antigenic profile of AbA cells

| | Primary | Primary | |
|---|---|---|---|
| | neonatal | neonatal | |
| | astrocytes (non- | astrocytes | |
| Antigen | transgenic) | (transgenic) | AbA cells |
| GFAP | XXX | XX (filamentous | X (diffuse |
| | (filamentous | pattern) | staining) |
| | pattern) | | |
| Nestin | X | X | X |
| Vimentin | X | X | XX |
| S100b | X | X | XXX |
| CX43 | X | X | XXX |
| GLT1 | XX | XX | — |
| GLAST | X | X | X |
| Glutamine synthase | XX | XX | XX |
| Ng2 | X | X | XX |
| Olig2 | — | — | — |
| A2B5 | — | — | — |
| ED1 | — | — | — |

Approximately 50% of the AbA and 20-30% of primary neonatal astrocytes were labeled in different degrees with antibodies to NG2 proteoglycan. Most AbAs were not stained with A2B5, indicating features of glial progenitors. However, AbA cells were negative for Olig2 that is known to be expressed by oligodendrocyte progenitor cells.

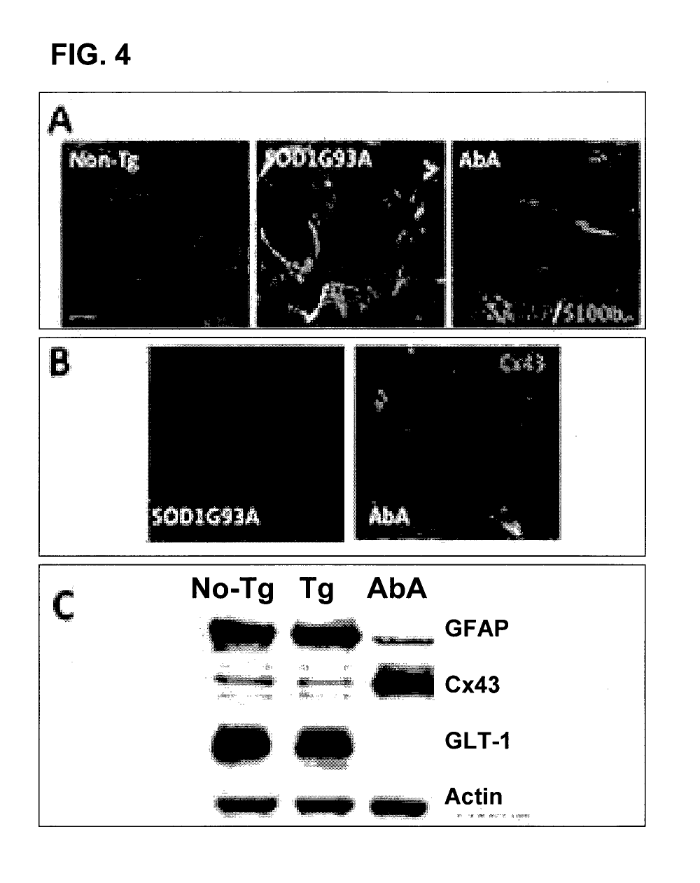

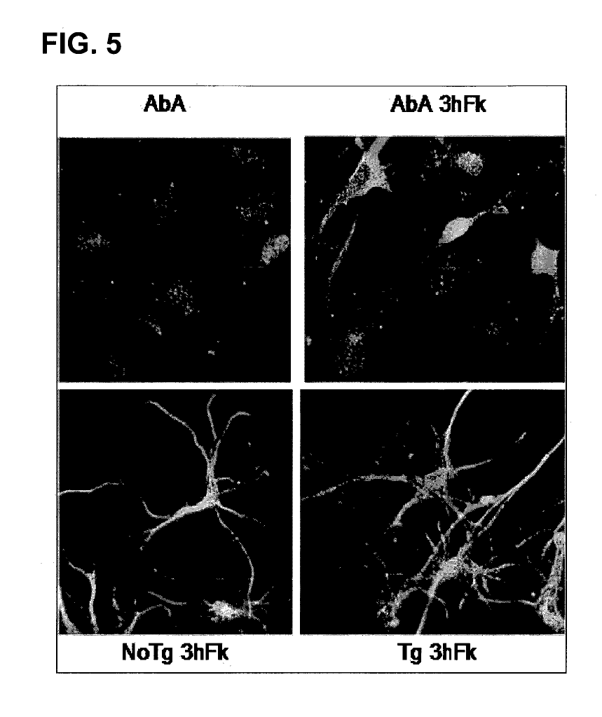

GFAP levels in AbA were decreased by 50% with respect of neonatal astrocytes and displayed a weak and diffuse cytoplasmic staining, as compared to the characteristic filament pattern in neonatal astrocytes (FIG. 3A). AbA displayed strong labeling to S100b, typically following a cytoskeleton-like pattern (FIGS. 3A and 4A). In comparison, NTg astrocytes displayed strong GFAP and weak S100b staining while Tg neonatal astrocytes displayed a mixed pattern of GFAP and S100b-positive astrocytes, some cells Co-Expressing both markers. AbA expressed high levels of Cx43 protein displaying a patchy distribution in the cell surface (FIGS. 3A and 4B). Compared to control astrocytes, the CX43 levels in AbA were increased by 10-15-folds, anticipating a key role in cell-cell communication or hemichannel formation. Compared to primary astrocytes, AbA cells failed to stain for the GLT1 glutamate transporter, as confirmed by Western blot (FIGS. 3B and 4C).

### Example 11

### Further Antigenic Characterization of Aba Cells

As depicted in FIG. 3A, AbAs displayed a weak and diffuse perinuclear GFAP labeling and intense staining for S100b, as compared to filamentous GFAP and weak S100b staining in neonatal astrocytes. GFAP levels in AbAs were decreased approximately 50% with respect to neonatal astrocytes, as estimated by western blotting (FIGS. 3B and 4C). To further determine the astrocytic nature of AbA, cultures were challenged with forskolin (10 uM), which promotes astrocyte differentiation in vitro. (Pollenz R S, McCarthy K D (1986) Analysis of cyclic AMP-dependent changes in intermediate filament protein phosphorylation and cell morphology in cultured astroglia. J Neurochem 47: 9-17.) As expected, forskolin induced process growth and increased GFAP staining in both neonatal astrocytes and AbAs, although the cell morphology was different between both groups (FIG. 3A).

To further determine the astrocytic nature of AbA, cultures were challenged with forskolin (10 uM), which promotes astrocyte differentiation in vitro. FIG. 2D shows forskolin induced process growth and increased GFAP staining in both neonatal astrocytes and AbA. However, the effect in AbA was restricted to a perinuclear GFAP accumulation and rapidly reversed after 6 h, suggesting an incapability to stay in a differentiated state.

AbA also expressed vimentin, glutamine synthase, and transgenic human SOD1 in comparable amounts to neonatal transgenic astrocytes (FIG. 3A-C). AbA expressed high levels of Cx43 protein, a connexin highly expressed in cultured astrocytes, displaying a cytoplasmatic and/or patchy distribution in the cell surface (FIG. 3A). (Giaume C, et al. (1991) Gap junctions in cultured astrocytes: single-channel currents and characterization of channel-forming protein. Neuron 6: 133-143.) Western blot analysis showed the CX43 levels in AbA were increased by 8-fold respect to control astrocytes, anticipating a key role of the protein in cell-cell communication or hemichannel formation (FIGS. 3B and 4C). (Evans W H, et al. (2006) The gap junction cellular internet: connexin hemichannels enter the signaling limelight. Biochem J 397: 1-14; Saez J C, et al. (2005) Connexin-based gap junction hemichannels: gating mechanisms. Biochim Biophys Acta 1711: 215-224; Bennett M V, et al. (2003) New roles for astrocytes: gap junction hemichannels have something to communicate. Trends Neurosci 26: 610-617.)

### Example 12

### Secreted Protein Profile of AbA Cells

In order to determine the proteins that are secreted by AbA cells and that could account for neurotoxicity, the inventors performed proteomic analysis of proteins secreted by AbA cells (secretome), both in a basal state and following activation with the pro-inflammatory stimuli LPS and interferon-gamma. The results show that AbA cells display a characteristic secretome profile, with a number of proteins that have not been previously described in other astrocytes types. This pattern might be relevant to identify AbA cells by analyzing biological fluids, such as CSF.

AbA cells were cultured in serum-free conditions during 24 h and the conditioned media were collected and concentrated. The samples were analyzed by two dimensional PAGE gels and the proteins were isolated from the spots and analyzed in a Maldi-tof-tof mass spectrometer. Table 2 describes proteins secreted by AbA cells in resting conditions.

TABLE 2
Secreted proteins by AbA cells in resting conditions

| Spot | Protein identification by Mass | Accession | | Mass |
|---|---|---|---|---|
| Number | Spectrometry | Number | Function | (KDa) |
| 1 | “transthyretin precursor” Rattus | P02767 | Transport | 15 |
| | norvegicus | | | |
| 2 | “transthyretin precursor” Rattus | P02767 | Transport | 15 |
| | norvegicus | | | |
| 3 | “transthyretin precursor” Bos Taurus | | | 15 |
| 4 | “hemoglobin, gamma” Bos Taurus | P02081 | | 15 |
| 5 | “Hemoglobin subunit alpha-2” Rattus | P01946 | | 15 |
| | norvegicus | | | |
| 6 | “profilin 1” Rattus norvergicus | P62963 | Cytoskeleton | 15 |
| 7 | “peptidylprolyl isomerase A | P10111 | Protein folding | 18 |
| | (cyclophilin A)” Rattus norvegicus | | | |
| 8 | “nucleoside diphosphate kinase B” | P19804 | Nucleotide | 17 |
| | Rattus norvegicus | | metabolism | |
| 9 | “smooth muscle protein SM22 | P31232 | Cytoskeleton | 19 |
| | homolog” - Rattus norvegicus | | | |
| 10 | Not identified | | | |
| 11 | “Human Cu—Zn Superoxide | P00441 | Oxidative | 16 |
| | Dismutase Mutant G93a” | | metabolism | |
| 12 | “transgelin” Rattus norvegicus | P31232 | Cytoskeleton | 22 |
| 13 | Not identified | | | |
| 14 | “transgelin” Rattus norvegicus | P31232 | Cytoskeleton | 22 |
| 15 | TIMP metallopeptidase inhibitor 2 | P30121 | Extracellular | 24 |
| | precursor Rattus norvegicus | | | |
| 16 | Not identified | | | |
| 17 | Not identified | | | |
| 18 | Not identified | | | |
| 19 | Not identified | | | |
| 20 | Not identified | | | |
| 21 | Not identified | | | |
| 22 | apolipoprotein A-I Bos taurus | | | 28 |
| 23 | apolipoprotein A-I Bos taurus | | | 28 |
| 24 | collagen, type I, alpha 2 precursor | P02466 | Extracellular | 130 |
| | Rattus norvegicus | | Matrix | |
| 25 | procollagen, type 1, alpha 1, isoform | P02454 | Extracellular | 72 |
| | CRA_a Rattus norvegicus | | Matrix | |
| 26 | collagen, type I, alpha 2 precursor | P02466 | Extracellular | 130 |
| | Rattus norvegicus | | Matrix | |
| 27 | ALB Bos Taurus | | | 69 |
| 28 | procollagen, type 1, alpha 1, isoform | P02454 | Extracellular | 72 |
| | CRA_a Rattus norvegicus | | Matrix | |
| 29 | Albumina Bos Taurus | | | 66 |
| 30 | Osteonectin/SPARC Rattus | P16975 | Extracellular | 34 |
| | norvegicus | | Matrix | |
| 31 | ALB protein | | | 69 |
| 32 | prepro complement component C3 | | | 187 |
| | Bos Taurus | | | |
| 33 | ALB bovina | | | 69 |
| 34 | Not Identified | | | |
| 35 | Precursor de la albumina bovina | | | 69 |
| 36 | Proteina ALB bovina | | | 69 |
| 37 | alpha-2-HS-glycoprotein precursor | | | 38 |
| | [Bos taurus] | | | |
| 38 | hypothetical protein LOC617667 [Bos | | | 46 |
| | taurus] | | | |
| 39 | serine proteinase inhibitor, clade A, | | | 46 |
| | member 1 precursor bovina | | | |
| 40 | alpha-2-HS-glycoprotein precursor | | | 38 |
| | Rattus norvegicus | | | |
| 41 | Precursor de la albumina bovina | | | 69 |
| 42 | Actina [Rattus norvegicus] | P60711 | Cytoskeleton | 58 |
| 43 | alpha-1 acid glycoprotein precursor | | | 23 |
| | [Bos taurus] | | | |
| 44 | Alfa fetuina Rattus norvegicus | P24090 | Extracellular | 38 |
| | | | Matrix | |
| 45 | Not Identified | | | |
| 46 | serine (or cysteine) proteinase | | Extracellular | 46 |
| | inhibitor, clade F, member 1 [Rattus | | Matrix | |
| | norvegicus] | | | |
| 48 | Biglycan Rattus norvegicus | P47853 | Extracellular | 42 |
| | | | Matrix | |
| 49 | Periostin Rattus norvegicus | Q62009 | Extracellular | 80 |
| | | | Matrix | |
| 50 | Perlecan Rattus norvegicus | Q05793 | Extracellular | 400 |
| | | | Matrix | |
| 51 | rCG30666, isoform CRA_b Rattus | EDL80841 | Extracellular | 46 |
| | norvegicus | | Matrix | |
| 52 | Ciclofilin F Rattus norvegicus | P29117 | Protein Folding | 28 |
| 53 | Insulin Growth Factor Binding | P12843 | Extracellular | 16 |
| | Protein Precursor 2 Rattus norvegicus | | | |
| 54 | Chain I Rat anionic Tripsin Complex | | | 11 |
| | with Bovine Pancreatic tripsin | | | |
| | inhibitor (BPTI) | | | |
| 55 | Cystatin C Rattus norvegicus | P14841 | Extracellular | 13 |

TABLE 3
Proteins secreted by AbA cells following activation by
inflammatory factors (LPS/interferon-gamma)

| | | Mass |
|---|---|---|
| Spot number | Identified protein by MS | (KDa) |
| 1 | Transtirretina Rattus Norvergicus | 15 |
| | Interferon-γhuman | |
| 2 | Matrix Gla Protein Rattus Norvegicus | 12 |
| 3 | Glutamate receptor, ionotropic kainate 4 | 18 |
| | Rattus Norvegicus | |
| 4 | S100 calcium binding protein A6 | 10 |
| | Rattus Norvegicus | |
| 5 | Chaperonin 10 Rattus Norvegicus | 10 |

### Example 13

### Distinctive Cx43/S100b+ Immunoreactivity with a Characteristic Distribution Nearby Motor Neurons in Degenerating Spinal Cord

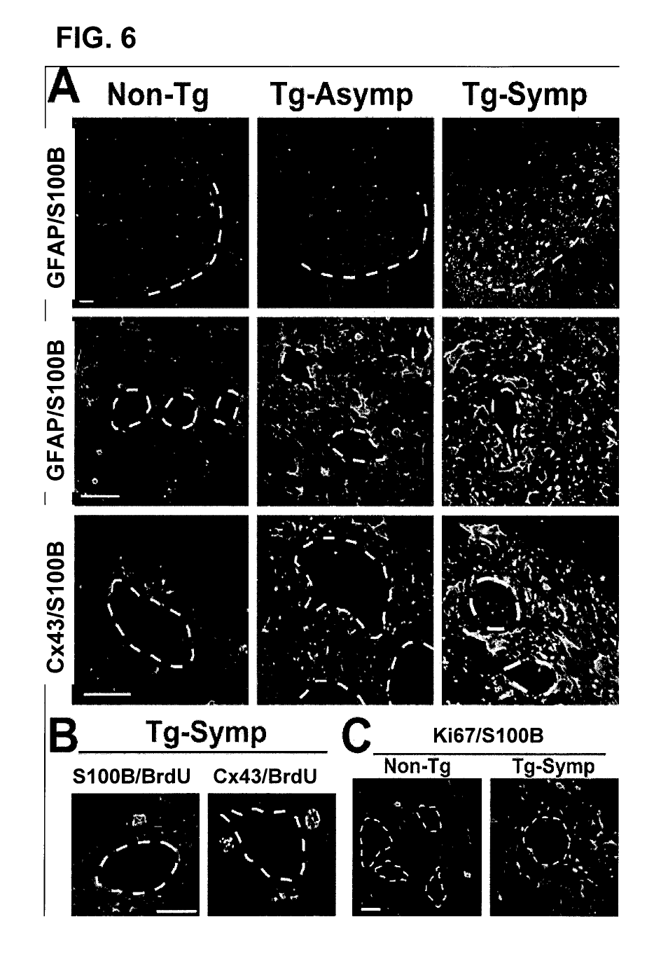

Characterization of AbA-like cells from the SOD1^{G93A} rat spinal cord demonstrates a distinctive immunoreactivity with a pathologically relevant distribution pattern. AbA-like cells were identified in the degenerating spinal cord as displaying distinctive Cx43/S100b+ immunoreactivity with a characteristic distribution nearby motor neurons and their efferent axons (FIG. 6). The emergence of aberrant astrocytes after disease onset may explain key aspects of ALS pathogenesis and defines a new diagnostic and therapeutic target for neurodegenerative diseases.

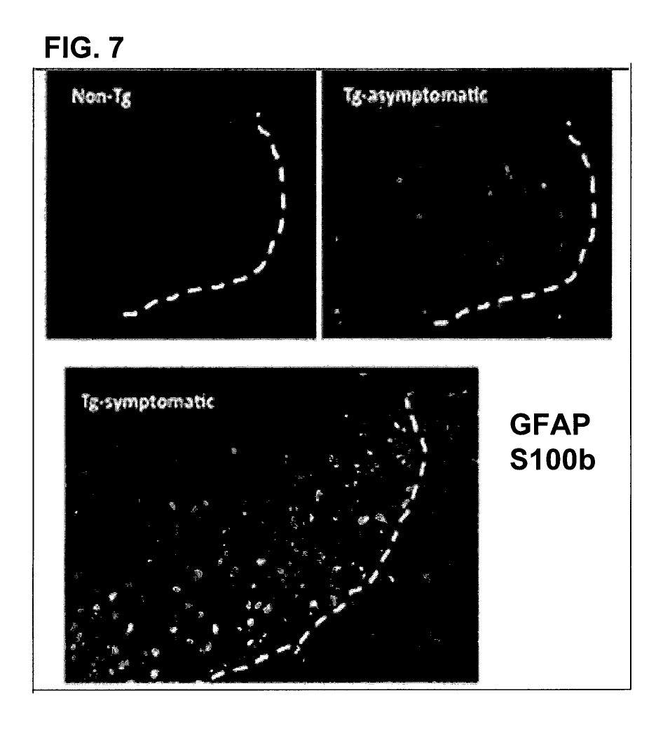

Further establishing whether cultured AbA cells are derived from endogenous astrocytes present in the degenerating spinal cord of SOD1^{G93A} rats, histological analysis was performed during the course of the disease using S100b/Cx43 as AbA cell markers. As depicted in FIG. 6A, S100b staining was low or moderate in non-Tg and Tg-Asymp rats. In contrast, S100b increased dramatically during the symptomatic stage of Tg rats. S100b immunoreactivity was mainly localized in a population of GFAP-expressing hypertrophic astrocytes bearing short processes (FIG. 7). Typically, these cells were observed in the ventral spinal cord nearby motoneurons and in the boundary between grey and white matter. Cx43 staining also increased dramatically in symptomatic rats and colocalized with S100b hypertrophic astrocytes. FIG. 6B shows perineuronal S100b or CX43-positive cells incorporated BrdU in symptomatic rats treated with BrdU 48 h before sacrifice, suggesting the AbA-like cell population actively proliferate in vivo.

### Example 14

### AbA Cells for High Throughput Drug Screening

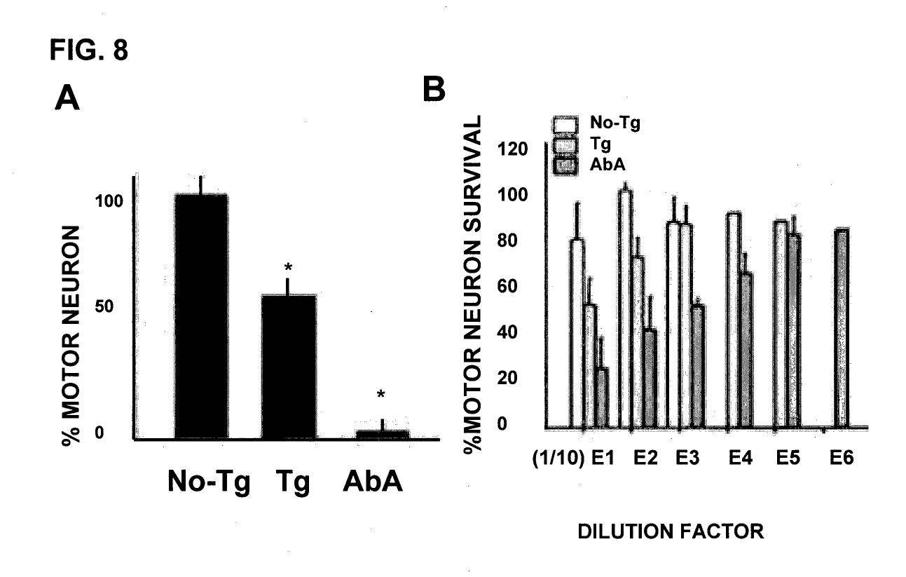

Remarkably, AbA cells exhibited marked toxicity when cocultured with motor neurons, exhibiting a 100-fold increased toxic activity in the conditioned medium as compared to primary neonatal SOD1^{G93A} astrocytes (FIG. 8). Since isolated AbA cells behave as a cell line, the availability of a homogenous cell population with neurotoxic properties can permit the screening for neuroprotective drugs, as discussed above.

### Example 15

### Primary Cultures of Neonatal Astrocytes

Heterozygous transgenic (Tg) SOD1^{G93A} and non-transgenic (non-Tg) astrocytes were prepared from spinal cords of 1 day-old pups according to the procedures of Saneto and De Vellis (1987) with minor modifications. (Cassina et al., 2002.) Astrocytes were plated at a density of 2×10⁴ cells/cm2 in 35 mm Petri dishes or 24-well plates and maintained in Dulbecco's modified Eagle's medium supplemented with 10% fetal bovine serum, HEPES (3.6 g/L), penicillin (100 IU/mL) and streptomycin (100 μg/mL). Astrocyte monolayers were >98% pure as determined by GFAP immunoreactivity and devoid of OX42-positive microglial cells.

### Example 16

### Motoneurons

Motoneuron cultures were prepared from embryonic day 15 (E15) wild type rat spinal cord by a combination of metrizamide-gradient centrifugation and immunopanning with the monoclonal antibody IgG192 against p75NTR. (Henderson C E, Bloch-Gallego E, Camu W (1995) Purification and culture of embryonic motorneurons. In: Cohen J ed. Neural cell culture: a practical approach (Oxford University Press, USA), pp 69-81.) Purified motoneurons were seeded on laminin coated substrate or onto astrocyte monolayers, depending on experimental protocols employed.

### Example 17

### Hippocampal Neurons

Hippocampi from E18 day embryos were dissected, received in Neurobasal (Gibco), containing 2% B27 (Gibco) and 1 mM glutamine, mechanically dissociated and passed through an 80 μm mesh. The isolated cells (300,000 cells/ml) were seeded onto plates covered with 0.1 mg/ml poly-D lysine and half of the media replaced days 3 after plating. At 5 DIV, cells were treated with conditioned media collected from primary nontransgenic and transgenic astrocytes, and AbA cells. Counting of viable cells was made after 24 h.

### Example 18

### Assessment of Motoneuron Survival

Motoneurons were plated at a density of 350 cells/cm² on 4 well multidishes or Lab-Tek (Nunclon) precoated with polyornithine-laminin. (Cassina et al., 2002.) Cultures were maintained in Neurobasal™ medium supplemented with 2% horse serum, 25 mM L-glutamate, 25 μM 2-mercaptoethanol, 0.5 mM L-glutamine, and 2% B-27 supplement (Gibco-Invitrogen). GDNF (1 ng/ml; Sigma) was added to the culture media to maintain the trophic support of motoneurons. After 24 hours in vitro, motoneurons were treated with dilutions of conditioned medium from nontransgenic and transgenic astrocytes, and from AbA cells. Survival was assessed after 48 hours by direct counting of all cells displaying intact neuritis longer than 4-cell bodies in diameter.

For co-culture experiments, motoneurons were plated on rat astrocyte (Tg and non-Tg) or AbA cell monolayers at a density of 300 cells/cm² and maintained for 48 h in complete L15 medium supplemented as previously described. (Cassina et al., 2002.) Motoneurons were recognized by positive immunostaining against p75NTR and labeling with a HRP conjugated antibody. Counts were performed over an area of 0.90 cm² in 24-well plates. (Cassina et al., 2002.) The mean density of motor neurons in control co-cultures was 84±3 cells/cm².

### Example 19

### Statistical Analysis

Statistical studies were performed using statistical tools of Origin 8.0. Descriptive statistic was used for each group and one-way ANOVA followed by Scheffe post hoc comparison if necessary were used among groups. All results are presented as mean±SEM, p<0.05 was considered significant.

### Example 20

### AbA Cells Specifically Induce Motor Neuron Death

Astrocytes carrying the SOD1^{G93A} mutation have been shown to induce a direct and specific deleterious effect on motor neuron survival. (Vargas et al. 2006; Di Giorgio et al., 2007; Nagai et al., 2007.) Thus, it seemed possible that AbAs might be the predicted cell type mediating adverse effects on motor neuron survival. To examine this possibility, immunopurified embryonic rat motoneurons were prepared and plated them on cultures of established monolayers of AbA cells or neonatal astrocytes. As depicted in FIG. 8, the number of motoneurons after 2 days in cocultures with AbAs was below 10% as compared with neuronal survival in cocultures with transgenic or nontransgenic neonatal astrocytes (FIG. 8A). These data suggest that AbAs exert a marked non-permissive environment for motor neuron growth and differentiation, remarkably more potent than other previously established in vitro systems for motor neuron culture.

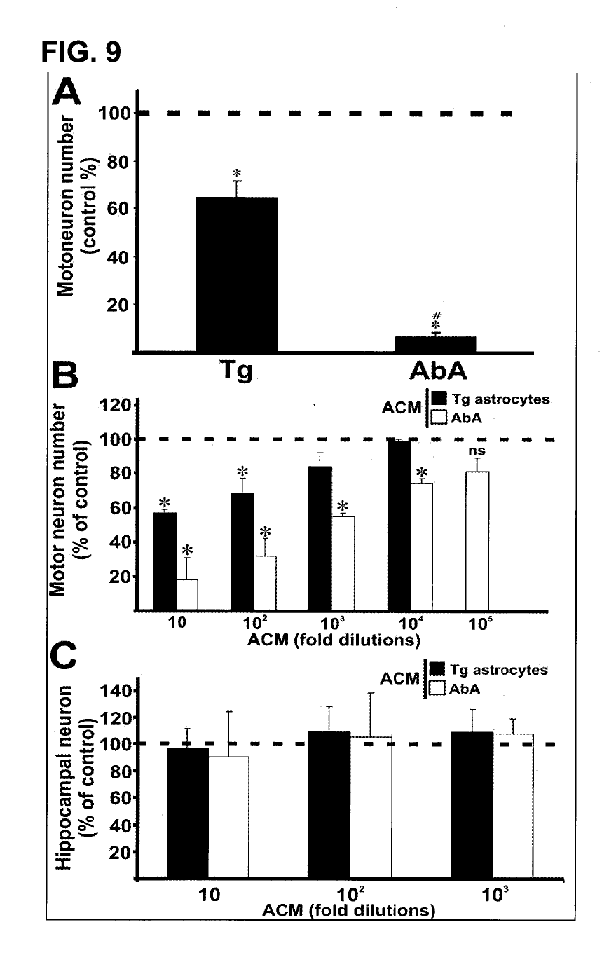

The mechanism underlying AbA cell toxicity was investigated and found to be mediated by soluble factors secreted into conditioned media (ACM), similar to a mechanism described for mutant primary astrocytes. (Nagai et al., 2007.) For this, embryonic motoneurons were cultured for 48 h on plates coated with laminin, poly-ornithine and maintained with GDNF as trophic factors. Then, increasing dilutions of ACM from AbAs or neonatal astrocytes were added to motoneuron cultures to assess their effect on survival. The numbers of motoneurons remaining after exposure to AbA conditioned medium (dilutions 1:10 to 1:10.000) were significantly lower than the permissive ACM from non-Tg neonatal astrocytes (FIG. 9B). Neonatal transgenic astrocytes significantly reduced motoneuron survival up to 1:100 dilutions as compared to a similar effect obtained with 1:10000 dilution of AbA conditioned medium. These data suggest that AbAs and transgenic astrocytes induce motoneuron death by secreting soluble factors, which apparently are produced in greater extent by AbA cells. In some instances, removal of serum present in AbA cell culture media will improve identification of factors associated with motoneuron death as secreted by AbA through elimination of trace amounts of serum proteins.

Towards establishing the specific of motorneuron toxicity as a result of secretory factors from AbAs, as has been reported for primary Tg astrocytes, the effect of conditioned media on primary cultures of embryonic hippocampal neurons was evaluated. (Nagai et al., 2007.) As shown in FIG. 9C, the number of neurons grown in 1:10 and 1:100 dilution of AbA conditioned medium was similar to ones grown on ACM from non-Tg astrocytes. Similar negative effects were observed in cultures of striatal neurons and sympathetic ganglion cells. Thus, AbAs and primary transgenic astrocytes shared the feature of specifically inducing motoneuron death.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the sources of AbA cells, isolation and culturing techniques used establish and manipulate AbA cells, diagnostic and therapeutic approaches related to ALS and other neurodegenerative diseases and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. An isolated cell having the ability to induce neuron death, wherein the isolated cell expresses at least one of the markers selected from the group consisting of GFAP, nestin, vimentin, S100β, CX43, GLAST, GLT1, glutamine synthase, Ng2, Olig2, A2B5, ED1, and combinations thereof.

2. The isolated cell of claim 1, wherein the isolated cell expresses at least one of the markers selected from the group consisting of GFAP, S100β, CX43, and combinations thereof.

3. The isolated cell of claim 1, wherein the isolated cell produces one or more secretory factors to induce the neuron death.

4. The isolated cell of claim 3, wherein the isolated cell secretes the one or more secretory factors into media to condition the media.

5. The isolated cell of claim 4, wherein the conditioned media exhibits at least 100 times more toxic activity to induce neuron death when compared to conditioned media from normal astrocytes.

6. (canceled)

7. (canceled)

8. The isolated cell of claim 1, wherein the isolated cell proliferates at a rate about two times that of normal astrocytes.

9. The isolated cell of claim 1, wherein the isolated cell is isolated from a mammal.

10. The isolated cell of claim 9, wherein the mammal has a SOD1^{G93} mutation.

11. The isolated cell of claim 10, wherein the SOD1^{G93} mutation is a SOD1^{G93A} mutation.

12. The isolated cell of claim 9, wherein the mammal is a transgenic rat having a SOD1^{G93A} mutation.

13. A culture comprising the isolated cell of claim 1.

14. A method of isolating cells having the ability to induce neuron death, comprising:
obtaining a tissue sample from a mammal;
disaggregating the tissue sample to extract cells;
plating the extracted cells on a culture surface; and
culturing the cells on the culture surface in culture medium, wherein the cells having the ability to induce neuron death are isolated and express at least one of the markers selected from the group consisting of GFAP, nestin, vimentin, S100β, CX43, GLAST, GLT1, glutamine synthase, Ng2, Olig2, A2B5, ED1, and combinations thereof.

15. The method of claim 14, wherein the isolated cells express at least one of the markers selected from the group consisting of GFAP, S100β, CX43, and combinations thereof.

16. The method of claim 14, wherein the isolated cells produces one or more secretory factors to induce the neuron death.

17. (canceled)

18. (canceled)

19. The isolated cell of claim 14, wherein the isolated cell proliferates at a rate about two times that of normal astrocytes.

20. The method of claim 14, further comprising placing a solid substrate in contact with the isolated cells.

21. (canceled)

22. The method of claim 14, wherein the tissue sample comprises motor cortex, brain stem, spinal cord, or skeletal muscle tissue from a mammal.

23. The method of claim 14, wherein the mammal has a SOD1^{G93} mutation.

24. The method of claim 23, wherein the SOD1^{G93} mutation is a SOD1^{G93A} mutation.

25. The method of claim 14, wherein the mammal is a transgenic rat having a SOD1^{G93A} mutation.

26. A method of treating a neurodegenerative disease comprising:
selecting a patient in need of treatment for a neurodegenerative disease; and
administering a compound capable of modulating the activity of an aberrant astrocyte to the subject for the neurodegenerative disease.

27. A method of detecting a neurodegenerative disease comprising:
obtaining a sample from a subject suspected of having a neurodegenerative disease;
measuring the level of a protein produced by an aberant astrocyte; and
comparing the level of the protein from the subject suspected of having a neurodegenerative disease to the level of the protein in a subject without the neurodegenerative disease, wherein a difference in the level of the protein detects the neurodegenerative disease.

28. (canceled)

29. A method of identifying a compound capable of modulating aberrant astrocyte activity, comprising:
providing a population of aberrant astrocytes;
contacting the population of aberrant astrocytes with a test compound;
measuring a parameter of the population of aberrant astrocytes, wherein the parameter is selected from the group consisting of proliferation, cytotoxicity, alterations in a protein function, or alterations in a protein expression, and combinations thereof; and
comparing the measured parameter from the population of aberrant astrocytes contacted with the test compound to a measured parameter from a population of aberrant astrocytes not contacted with the test compound, wherein a difference in the measured parameter identifies the compound capable of modulating aberrant astrocyte activity.

30. (canceled)
